(19) United States
(12) Patent Application Publication
(10) Pub. No.: US 2002/0012694 A1
(43) Pub. Date: Jan. 31, 2002

(54) TRANSDERMAL ADMINISTRATION OF MENT

(76) Inventors: ALFRED J. MOO-YOUNG, HASTINGS-ON-HUDSON, NY (US); YUN-YEN TSONG, NORTH CALDWELL, NJ (US)

Correspondence Address: LERNER, DAVID, LITTENBERG,, KRUMHOLZ & MENTLIK, 600 SOUTH AVENUE WEST, WESTFIELD, NJ, 07090, US

(21) Appl. No.: 09/154,287

(22) Filed: Sep. 16, 1998

Related U.S. Application Data
(60) Provisional application No. 60/059,301, filed on Sep. 17, 1997.

(51) Int. Cl. A61K 9/00; A61K 9/70; A61F 13/00

(52) U.S. Cl. 424/449; 424/400

(57) ABSTRACT

The present invention relates to transdermal dosage forms for delivery of androgens.

### CROSS-REFERENCE TO RELATED APPLICATIONS

The present application claims benefit of United States Provisional Patent Application 60/059,301, filed Sep. 17, 1997, the disclosure of which is hereby incorporated by reference herein.

### FIELD OF THE INVENTION

The present invention relates to the fields of medicine and pharmacology and specifically to the production of transdermal devices for the administration of MENT and transdermal methods of administering MENT.

### BACKGROUND OF THE INVENTION

Due to the obvious shortcomings of physical barrier techniques, many have sought a chemical solution to male contraception. But the problems of developing a suitable, chemically based male contraceptive are significant. Sterilants such as LHRH can be administered and they will effectively reduce a male's sperm count. However, there may be an accompanying loss of male sexual function. Therefore, there has been proposed various subcutaneous implantable systems for administering both a sterilant and an androgen. See U.S. Pat. No. 5,733,565. These systems are effective and can provide generally long-term, maintenance-free male contraception.

But these systems are not without their drawbacks as well. Some people have an aversion to subcutaneous implants. Implanting, extracting and reimplanting devices require inconvenient doctor's visits. And, while minimally invasive, all of these are surgical procedures. As a result, some subjects will be noncompliant. Others will refuse this form of contraception as an alternative. And there is always a risk of secondary infection.

Of course, the dosage form itself is not the only problem. Selecting an appropriate androgen is often a significant issue as well. For example, testosterone is often used for androgen replacement therapy. However, the potency of testosterone is limited, requiring large doses to be administered. Testosterone is also 5α reduced to a metabolite known as Dihydrotestosterone (“DHT”). DHT is very active on the prostate and can cause abnormal prostate growth.

Therefore, there exists a need for convenient and effective, nonsurgical methods for administering potent, safe and effective androgens to subjects in need thereof. These objectives are satisfied by the present invention.

### SUMMARY OF THE INVENTION

It has now been discovered that, contrary to the teachings of the art, it is possible and advantageous to administer androgen transdermally. While testosterone provides significant problems for transdermal applications, other androgens and in particular, 7α-methyl-19-nortestosterone (“MENT”) cannot only be administered transdermally, but can offer great advantage. Therefore, the present invention relates to a transdermal dosage form. The transdermal dosage form can be an ointment, cream, gel, powder, transdermal patch, lotion, spray or the like. The dosage form is produced from a non-5α-reducible androgen provided in at least a therapeutically effective amount and is disbursed within a pharmaceutically-acceptable transdermal carrier. The carrier can be an ointment based, gel based, cream based, lotion based, powder based, spray based or a transdermal patch into which the androgen is formulated.

In preferred aspects of the present invention, the non-5α-reducible androgen is a 7α-modified androgen and in particular, MENT.

Indeed, in a particularly preferred embodiment of the present invention, there is provided a transdermal dosage form which includes an amount of between about 0.5 and about 10 mg of MENT disbursed in the pharmaceutically acceptable transdermal carrier. The MENT is provided in an amount of between about 0.5 to about 90% by weight relative to the weight of the carrier. In a particularly preferred embodiment, the transdermal dosage form can include between about 0.5 and about 10 mg of androgen, as previously discussed, for each day of application. Therefore, if a patch were to be applied for a total of three days, it could contain between about 1.5 and about 30 mg of, for example, MENT.

The transdermal application of an androgen is ideal for, inter alia, contraception and androgen replacement therapies. Transdermal systems are easy for the subject to use, enhancing compliance. Transdermal systems can generally be produced inexpensively and in a number of different relevant formats aiding the appeal and level of access to these types of therapies. While periodic monitoring by a medical professional is still advisable, doctor's visits are not needed for continued therapy. As a noninvasive drug delivery technique, the risks of side effects are greatly reduced. Moreover, unprecedented control is placed in the hands of the subject.

It is not enough, however, to merely propose the transdermal application of androgens in general. It has been found that the transdermal administration of MENT provides significant advantages in terms of safety and efficacy in transdermal formats.

It has been discovered that the flux, i. e. the amount of drug capable of penetrating a known surface area of skin over a given period of time, for MENT can be as much as twice that of testosterone or more depending upon the particular transdermal formulation. For a purely hypothetical example, if a transdermal patch were formulated to provide 1 mg of MENT to a subject per day and the patch were 2.5 cm by 2.5 cm, it could take as much as 48 hours, or even more for the same amount of testosterone to permeate the same skin area. To obtain a comparable flux, two 1 cm by 1 cm patches containing 1 mg each of testosterone would be necessary. Even ignoring the additional cost of the drug, it would cost at least twice as much to treat the subject with testosterone as it would with MENT under these circumstances. Furthermore, the application of two patches or one larger patch is less convenient and less desirable potentially reducing compliance.

Merely delivering the same quantity of testosterone over the same period is not useful, as testosterone is between 5 and 10 times less potent than MENT. Therefore, to obtain the same level of biological activity, i.e. to have the same bioavailability, one would need as much as 10 mg of testosterone to equal the therapeutic efficacy of 1 mg of MENT. Providing ten 2.5 cm by 2.5 cm patches therefore would be necessary to provide the same potency as that obtained from one 1 mg patch of MENT. Of course, because of the flux of testosterone, those ten patches will still require 48 hours to deliver their full dose. Therefore, to obtain true bioequivalency, i.e. the same biologically effective dose in the same period of time, twenty 2.5 cm by 2.5 cm patches would be required. As a result, instead of a 1″ by 1″ patch, a subject would have to cover a 5″ by 4″ area of skin.

Even this example is not truly representative. Skin contains various enzymes which can metabolize androgens applied transdermally. The greater the surface area and the period of time of administration, the greater the degree to which those metabolic enzymes can work. Therefore, it would be more accurate to state that at least about twenty 2.5 cm by 2.5 cm patches would be necessary to provide comparable levels of testosterone over a comparable period of time. Indeed, the situation could be much worse. The transdermal application of MENT would involve the once-a-day application of a 1″ by 1″ patch while the use of testosterone would require a good deal more. Not only would the use of testosterone patches be inconvenient, it would also be very costly.

Moreover, since testosterone is 5α reduced to DHT, trying to match MENT's efficacy by increasing testosterone administration will only result in an increase in significant side effects such as, for example, overstimulating the prostate. Testosterone, MENT and other 7α-modified androgens are also powerful steroid based drugs and to the extent possible, reducing the degree of exposure to such steroids is desirable.

It has been found that MENT can be administered transdermally to provide steady-state blood levels which are sufficient for therapeutic purposes. It has also been found that MENT allows for the construction of delivery vehicles which are cost-effective, efficient and compliance enhancing.

### BRIEF DESCRIPTION OF THE DRAWINGS

FIG. 1 illustrates the “in vitro” permeation of testosterone (“T”) and MENT across rat skin as measured as a function of the concentration (microgram/mL) versus time for the gel described in example 2.

FIG. 2 illustrates the steroid flux measured in micrograms/cm²/hours for MENT and T across rat skin of the gel described in example 2.

FIG. 3 illustrates the permeation profile (flux) of MENT and T across rat skin for the gel formulation described in example 1 (A)(1)(KY jelly).

FIG. 4 illustrates the permeation profile (flux) of MENT and T across rat skin from the gel described in example 1 (A) (2) (pharmacist value lubricating jelly).

FIG. 5 illustrates the permeation profile (flux) of MENT and T across rat skin from a transdermal patch described in example 1 (B).

FIG. 6 illustrates the permeation profile (flux) of MENT and T across rat skin from a cream formulation described in example 1 (C)(1) (cream base A).

FIG. 7 illustrates the permeation profile (flux) of MENT and T across rat skin from a cream formulation described in example 1 (C)(2) (cream based B).

FIG. 8 illustrates the permeation profile (flux) of MENT and T of a cream formulation discussed in example 1 (C)(3).

FIG. 9 illustrates the permeation profile (flux) of MENT and T across rat skin from gel O described in example 1 (D).

FIG. 10 illustrates the permeation profile (flux) of MENT and T across rat skin from gel D described in example 1 (D).

FIG. 11 illustrates the permeation profile (flux) of MENT and T across rat skin from gel F described in example 1 (D).

FIG. 12 illustrates the permeation profile (flux) of MENT and T across rat skin from gel P described in example 1 (D).

FIG. 13 illustrates the permeation profile (flux) of MENT and T across rat skin from gel T described in example 1 (D).

FIG. 14 illustrates MENT blood levels following topical application to rabbit skin as a function of concentration (ng/mL) versus time for gel formulation O at a dosage of 0.4 mg MENT/0.2 mL gel.

FIG. 15 illustrates MENT blood levels following topical application to rabbit skin as a function of concentration (ng/nl) versus time for gel formulation O at a dosage of 0.8 mg MENT/0.4 mL gel.

FIG. 16 illustrates MENT blood levels following topical application to rabbit skin measured as a function of concentration in ng/mL versus time for gel formulation F at a dosage of 0.4 mg/0.2 mL gel.

FIG. 17 illustrates MENT blood levels following topical application to rabbit skin measured as a function of concentration in ng/mL versus time for gel formulation F at a dosage of 0.8 mg/0.4 mL gel.

FIG. 18 illustrates MENT blood levels following topical application to rabbit skin measured in concentration of MENT ng/mL versus time for gel formulation T at a dosage of 0.4 mg/0.2 mL gel.

FIG. 19 illustrates MENT blood levels following topical application to rabbit skin measured in concentration of MENT ng/mL versus time for gel formulation T at a dosage of 0.8 mg/0.4 mL gel.

FIG. 20 illustrates the Brookfield viscosity of a MENT gel formulation prepared in accordance with examples 1 and 2 (Formulation T), using 3 different spindles.

FIG. 21 is a cross-sectional, planer view of a transdermal patch in accordance with one aspect of the present invention.

### DETAILED DESCRIPTION OF PREFERRED EMBODIMENT

A pharmaceutically-acceptable, transdermal carrier is an ointment base, cream base, lotion base, salve base, gel base, powder base or carrier material used for the construction of a transdermal patch. The term can also include for example, physical materials such as gauze, cloth and the like. The term “dispersed” includes dissolved, distributed, emulsified, homogeneously mixed, suspended and the like.

The androgen used in accordance with the present invention is a non-5α-reducible androgen. Testosterone is excluded by this definition as it is a 5α-reducible androgen, and as such, can produce higher levels of adverse side effects than equivalent potencies of other androgens as described. Non-5α-reducible androgens include, without limitation, 7α-modified-androgens. Examples of these include 7α-alkyl-androgens such as 7α-methyl-14-dehydro-19-nor-testosterone (CDB-868B), 7α-methyl-17αβ-propiony-loxy-D-homoestra-4, 16, dien-3-one (CDB 2322A) and 7α-methyl-19-nortestosterone (MENT) and their pharmaceutically acceptable salts. See Kumar et al., “The Biological Activity of 7α-Methyl-19-Nortest-osterone Is Not Amplified in Male Reproductive Tract as is That of Testosterone,” Endocrinology, Vol. 130, No. 6, pgs. 3677-3683 (1992). The most preferred androgen is MENT, its acetate, MENT Ac and related compounds. However, it has been found that the flux of MENT is generally greater than that of MENT Ac so MENT is preferred for many of the transdermal techniques and devices described herein.

Other androgen compounds useful in the method of the invention are testosterone derivatives having a non-hydrogen substituent in the 6α or 7α position. As used in the application, the term testosterone derivatives encompasses compounds having the basic four ring structure of testosterone, optionally modified at the 3, 5, 9, 11, 17 or 19 positions. Examples of such compounds include:

7-α-methyl testosterone,

7-α-methyl-11β-hydroxytestosterone,

7-α,17-dimethyltestosterone,

7-α,17-dimethyl-11β-hydroxytestosterone,

7-α,17-dimethyl-19-nortestosterone,

7-α,17-dimethyl-11β-hydroxy-19-nortestosterone,

6-α-methyl testosterone,

6-α-methyl-19-nortestosterone,

6-α-methyl-11β-hydroxytestosterone,

6-α,17-dimethyltestosterone,

6-α,17-dimethyl-11β-hydroxytestosterone,

6-α,17-dimethyl-19-nortestosterone, and

6-α,17-dimethyl-11β-hydroxy-19-nortestosterone

The 7α-methyl compounds for use in the invention can be prepared as described in U.S. Pat. No. 3,341,557 which is incorporated herein by reference. Synthesis of the other compound identified herein have also been described in the literature.

The transdermal dosage forms of the present invention will have application in a wide range of indications including, without limitation, androgen replacement therapy, contraception, primary hypogonadism, testicular failure, baldness, aging, loss of bone mass, muscle wasting and cachexia, BPH, and prostate cancer. Therefore, the dose of androgen necessary can vary significantly. Furthermore, the potency, bioequivalence and bioactivity of the androgens useful for these indications can vary significantly. That can also have a dramatic effect on dosing. Other factors including the size, health and biochemistry of the subject also play a significant role in dosing.

The type of transdermal dosage form and the androgen used play a large role in formulation, if not the actual dose to be administered. It must be remembered that every microgram of drug formulated into the dosage forms of the present invention does not necessarily make across a subjecfs skin and into their circulatory system. Therefore, in certain instances, it may be necessary to formulate with an excess of androgen to ensure that the correct amount of drug is delivered across the skin in a bioavailable form. Certain dosage forms may also be limited in terms of size, solubility, flux and drug capacity. These factors, plus the size of the desired dose and the time over which that dose is to be administered can all have an effect on the amount of androgen in the dosage form.

Therefore it is necessary to distinguish between the dose, that amount which is actually bioavailable over a certain period of time, and the amount used to formulate the dosage form. These amounts can be equal. However, the amount used to formulate the dosage form is more often in excess of the desired dose. Moreover, unless otherwise indicated either specifically or by context, references to an amount of an androgen dose generally refer to the amount that is bioavailable over a 24 hour period. For example, a transdermal patch may be formulated with 2 milligrams of MENT. The flux of MENT coupled with the area of the patch may dictate that in 24 hours, only 1.25 milligrams is dispensed. And of that, only 1 milligram is actually bioavailable. That 1 milligram, is never present all at once. The total which is bioavallable over the course of 24 hours is 1 milligram. This is therefore the amount of bioavailable MENT. In this case, 1 milligram of MENT over 24 hours can provide steady-state blood levels of over 1.0 nmol/L throughout the day.

Generally the amount of androgen that is bioavailable can be determined in vitro by the methods of determining flux as described herein or in vivo by actual blood tests from the subject using known methods. The amount of androgen administered transdermally can be adjusted on a subject by subject basis to provide optimal results. Using the prior example, a certain transdermal patch would be expected to provide 1 milligram of MENT in bioavailable form to a subject over 24 hours. However, blood tests could indicate that a particular subject is not responding at this level. Alternatively, because of this subject's metabolic system, less than the full 1 milligram is actually bioavailable. Either situation could be addressed by reformulating the patch to administer more androgen and/or by expanding the surface area of the patch to account for the need to deliver a certain amount of androgen at the flux for that androgen from that dosage form.

The amount of androgen delivered in bioavailable form will therefor generally range from about 50 micrograms to as much as about 8 milligrams over the course of a 24 hour day. That could mean that significantly more androgen, i.e. 10 mg/day/dose, is formulated into a daily dosage form. Therefore, if a patch was to deliver 1 mg of MENT in bioavailable form each day for 3 days before being replaced, but an excess of 1 mg/day was needed in the patch to obtain the desired amount in bioavailable form, then the patch would be formulated with 2 mg of MENT per day for each day of use for a total of 6 mg. More preferably, the amount of androgen which is bioavailable over a 24 hour period can range from between about 100 micrograms to about 2 milligrams and most preferably from between about 400 to about 1600 micrograms. The term “therapeutically-effective amount” is intended to mean the bioavailable amount of androgen which is sufficient to produce a desired response.

Finally, while the dose and dosing will usually be discussed in terms of administration over a 24 hour period, that is not a limitation. A gel formulation might be capable of providing a great deal of androgen across the skin in a relatively few hours (1-4 hours). Peak serum levels could be reached very quickly. So long as the dose is calculated to provide serum levels at or above some minimum target amount, the therapeutic effect of the dose should be maintained. Similarly, a transdermal patch may be used for 2, 3 or 4 days, or even a week before being replaced.

Transdermal dosage forms in accordance with the present intention can take any number of forms. These formulations can include powders, cosmetics, ointments, gels, creams, lotions, salves and the like. Androgen can be formulated into transdermal patches by mixing the androgen -with a material which is itself adhesive or by adhering a non-adhesive drug containing reservoir or carrier to the skin of the subject using a backing material having adhesive at its peripheral, skin facing surface. Hydrogel materials, which are adhesive or non-adhesive, can be used for this purpose. Drugs can also be formulated into adhesive and non-adhesive bandages.

The topical dosage forms of the present invention are prepared according to procedures well known in the art and may contain other active ingredients (also referred to herein as the “androgen”). For example, the androgen may be formulated into a preparation suitable for topical administration in an ointment, lotion, gel, cream, topical spray and/or powder.

Ointments and creams may, for example, be formulated with an aqueous or oily base with the addition of suitable thickening, gelling and/or emulsifying agents. Such bases may thus, for example, include water and/or an oil such as petrolatum, liquid paraffin or a vegetable oil such as peanut oil or castor oil. Thickening agents which may be used according to the nature of the base include soft paraffin, aluminum stearate, cetostearyl alcohol, polyethylene glycols, woolfat, hydrogenated lanolin, beeswax, etc. Emulsifying agents may include, for example, PEG monosterate, lauril sulfate, Tween 80, sodium deoxycholate, Brji 30, Myrj 45, etc.

Lotions may be formulated with an aqueous or oily base and will in general also include one or more of the following, namely, stabilizing agents, emulsifying agents, dispersing agents, suspending agents, thickening agents, coloring agents, perfumes and the like.

Gels may be produced using well known techniques from conventional pharmaceutically acceptable gelling agents including, without limitation, modified celluloses such as methyl cellulose, hydroxy methyl cellulose, hydroxy propyl methyl cellulose, starch, modified starches, natural and synthetic gums including tragatanth, guar, acacia, carrageenan and the like, gelatin, sodium alginate, PVP, polyvinyl alcohol, and CARBOPOL's available from CRODA, Inc. of Edison, N.J.

Powders may be formed with the aid of any suitable powder base, e.g. talc, lactose, starch, etc. Drops may be formulated with an aqueous base or non-aqueous base also comprising one or more dispersing agents, suspending agents, solubilizing agents, etc.

The pharmaceutical compositions according to the invention may also include one or more preservatives or bacteriostatic agents, e.g. methyl hydroxybenzoate, propyl hydroxybenzoate, chlorocresol, benzalkonium chlorides, etc. The compositions according to the invention may also contain other active ingredients such as antimicrobial agents, particularly antibiotics.

The proportion of androgen in the compositions according to this aspect of the invention depends on the precise type of formulations to be prepared but will range of from 0.5% to 90% by weight. Generally, however, for most types of preparations advantageously the proportion used will be within the range of from 1.0 to 80% and more preferably 5.0-50% by weight.

Formulating dosage forms in accordance with the present invention may be as simple as measuring a desirable amount of a specific androgen and homogeneously blending the androgen with a carrier or base, such as a cream, lotion, gel, etc., as described above. In the context of a gel, the androgen can be introduced prior to gel formation or physically blended with the gel thereafter. The androgen could also be blended with a known amount of, for example, a drug releasing adhesive before the adhesive has formulated into a patch and/or dried, cross-linked, or the like as discussed herein. Often however the androgen will have to be solubilized in a solvent prior to formulation with the carrier. Formulating the androgen in a solvent would allow the material to be conveniently homogeneously mixed with certain bases such as adhesive materials, creams and ointments. Use of a solvent may also help in emulsification and/or absorption on for example, gauze patches used in an adhesive style bandage.

Solvents useful in formulating the transdermal dosage forms of the present invention are non-toxic, pharmaceutically acceptable substances, preferably liquids. The solvent is preferably an alcohol including polyhydric alcohols or combination of polyhydric alcohols. The term polyhydric alcohol means any organic polyalcohol and includes dipropylene glycol, propylene glycol, polyethylene glycol, glycerin, butylene glycol, hexylene glycol, polyoxyethylene, polypropylene glycol, sorbitol, ethylene glycol, and the like. Polyhydric alcohols may include those having 2 to 6 alcoholic hydroxyl groups.

Other suitable solvents include fatty acids such as oleic acid, linoleic acid, capric acid and the like, as well as fatty esters or alcohols. Further suitable solvents include other non-toxic, non-volatile solvents commonly used in dermal or transdermal compositions for dissolving like compounds.

Although the exact amount of the solvent used in these formulations depends on the nature of other components, and therefore cannot be stated in general terms, the proportion may range from about 5 to about 70 weight percent based on the whole composition. Preferably the androgen is substantially dissolved in the solvent so that when mixed with the adhesive or other carrier materials, the androgen is dispersed and/or dissolved.

Solvent selection for a single androgen or a combination of androgens in either the free base form or in a salt or derivative form, depends in part on the form of the androgen. Solvents for the salt forms are generally polar organic solvents. Polar organic solvents are preferably polyhydric alcohols, as discussed above. Various other solvents include cyclic ketones such as 2-pyrrolidone; N-(2-hydroxyethyl) pyrrolidone, N-methylpyrrolidone, 1-dodecylazacyclo-heptan2-one and other n-substituted alkyl-aza-cycloalkyl-2-ones (azones) dimethylformadide, and dimethylsulfoxide.

Other suitable solvents for the free base form of the androgen are cell envelope disordering compounds known to be useful in topical pharmaceutical preparation, which compounds are thought to assist in skin penetration by disordering the lipid structure of the stratum corneum celiienvelopes. See U.S. Pat. No. 5,332,576, the text of which is hereby incorporated by reference.

Another particularly useful transdermal dosage form for delivering androgens in accordance with the present invention are transdermal patches. While there are an almost infinite variety of transdermal patches which can be used, there are many which share a number of common elements. For example, many patches useful in accordance with the present invention include an occlusive outer surface or backing layer. The backing layer is preferably a thin film or sheet. In many instances, because of the area of skin to which the device is to be attached, the device, and therefore the backing layer, is flesh colored for cosmetic reasons. But that need not be the case. Preferably, it is a clear polyester layer, which is occlusive with respect to the active agent or drug, which in this case includes at least one androgen, but it can be dyed various colors, or include printed matter thereon. The backing layer normally provides support and a protective covering for the patch device.

The backing layer is preferably made of a material or combination of materials that is substantially impermeable to the drug containing layer or layers with which it can be in contact, i.e., to the drug carrier layer and the androgen and, possibly other active ingredient(s) contained therein, the adhesives, etc. However, a primary objective is to prevent seepage of the active ingredient through the backing layer of the device so, if the backing layer is coated on the surface in contact with the remainder of the device with an adhesive layer that is active ingredient impermeable, this impermeable adhesive layer will perform this purpose even if the backing layer is not totally impermeable to the active ingredient. Thus, it is not necessary in all instances that the backing layer be impermeable to the active ingredient, although in most instances it normally is, and when it is not a layer providing this barrier function, such as an active ingredient impermeable adhesive layer, will be situated between the backing layer and the carrier layer. By substantially impermeable, it is meant that the other components in contact with the backing layer or component under consideration will not appreciably permeate through such layer or component for the normal period of use and storage of the device.

The actual material used for the backing layer will depend on the properties of the materials in contact therewith. Some suitable materials include, for example, cellophane, cellulose acetate, ethyl cellulose, plasticized vinyl acetate-vinyl chloride copolymers, ethylene-vinyl acetate copolymer, polyethylene terephthalate, nylon, polyethylene, polypropylene, polyvinylidene chloride (e.g., SARAN), paper, cloth and aluminum foil. The material used is preferably impermeable to the active ingredient. The material which forms this backing layer may be flexible or non-flexible. Preferably, a flexible backing layer is employed to conform to the shape of the body member to which the device is attached.

Preferably, the material which forms the backing layer is a film or a composite film. The composite can be a metallized (e.g., aluminized) film or a laminate of two or more films or a combination thereof For example, a laminate of polyethylene terephthalate and polyethylene or a polyethylene/metallized polyethylene terephthalate/polyethylene laminate can be employed. The preferred polymers include polyethylene, polypropylene, polyvinyl chloride, polyethylene terephthalate and polyvinylidene chloride (SARAN).

The backing layer may be affixed to the androgen containing carrier layer(s) either directly, where the carrier layer is both adhesive to the skin and the backing layer, or by an adhesive layer. Where an adhesive layer is used, as previously discussed, the adhesive layer may be active ingredient (androgen) impermeable to prevent seepage of the androgen from the carrier layer to the backing layer, and should be androgen impermeable when the backing layer is not. The adhesive layer and the backing layer may extend peripherally beyond the carrier layer about the entire periphery thereof so as to create an extended peripheral area of the backing layer with the adhesive layer peripherally extending beyond the carrier layer coextensively with the extended peripheral area of the backing layer. Therefore, another purpose of the adhesive layer can be to secure the device to the skin or mucosa.

Any adhesive capable of providing adhesion of the backing layer to the carrier layer and/or the backing layer to the skin will be suitable for use. Preferably, the adhesive layer is a pressure-sensitive adhesive suitable for contact with the skin or mucosa, e.g., dermatologically acceptable. Active ingredient (androgen) impermeable adhesives are typically coated onto the carrier or backing layer in liquid form. The liquid form of the adhesives are obtained either by dissolution or suspension of the adhesive components in a liquid vehicle or emulsion or by heating a thermoplastic adhesive above its melt temperature. The adhesive layer is then either dried by evaporation of the liquid vehicle or emulsion or hardened by cooling thermoplastic material below its melt temperature. Active ingredient impermeable adhesives are thus defined as being impermeable to the active ingredient when the adhesive layer is substantially dry or hardened.

Examples of suitable pressure sensitive adhesive materials for use in the present invention as the active ingredient impermeable adhesive layer include some natural rubber and synthetic rubber adhesives and cross-linkable laminating adhesives. Examples of suitable natural rubber adhesives include R-1072 from B. F. Goodrich Co., No. 735 from C. L. Hathaway, and No. 5702 from Evans St. Clair. Examples of synthetic rubber adhesives include Jowatherem 270-00 and Jowatherem S-3202 from Jowat Corp. and 70-9416 from National Starch. Other suitable laminating adhesives include the Dow Corning laminating silicone adhesives and the Lord Corporation Tycel 7900 series laminating adhesives. The adhesives most impermeable to most active ingredients are cross-linkable laminating adhesives, which are well-known to those of ordinary skill in the art.

When utilizing pressure-sensitive adhesives, as the thickness of the adhesive layer affixing the backing layer to the carrier layer increases, the impermeability of the adhesive layer to the active ingredient also increases. To provide active ingredient impermeability to the adhesive layer, the thickness of the active ingredient impermeable adhesive layer is that thickness that provides sufficient impermeability to the active ingredient (and if necessary, to the other components of the device with which the impermeable adhesive layer is in contact) so that the active ingredient does not seep out of the device as explained above. Typically, to obtain active ingredient impermeability, the impermeable adhesive layer joining the backing layer to the carrier layer will have a thickness between about two and about five mils, and preferably will have a thickness of about two mils. Cross-linkable pressure-sensitive adhesives provide even greater impermeability of the adhesive layer to active agents and enhancers. By increasing the cross-link density of the adhesive layer, an even greater barrier to active agent diffusion is provided.

The patches of the present invention may also include an active ingredient permeable adhesive layer between the carrier layer and the skin or mucosa of the subject, joining the device thereto. Certain embodiments may utilize a plurality of such active ingredient permeable adhesive layers. For example, an active ingredient permeable adhesive layer can be used to affixes a rate-controlling polymer layer to a surface of the androgen containing carrier layer. The device is then affixed to the skin or mucosa of the subject by a second active ingredient permeable adhesive layer which is applied to the surface of the rate-controlling polymer layer opposite to carrier layer.

At least the active ingredient (androgen) permeable adhesive layer that joins the device to the skin or mucosa of the subject is preferably dermatologically acceptable. Each active ingredient permeable adhesive layer is also preferably a pressure-sensitive adhesive. Any of the well-known, dermatologically acceptable, pressure-sensitive adhesives which permit drug migration there through can be used in the present invention. Some suitable permeable adhesives include acrylic or methacrylic resins such as polymers of alcohol esters of acrylic or methacrylic acids and alcohols such as n-butanol, isopentanol, 2-methylbutanol, 1-methyl-butanol, 1-methyl-pentanol, 2-methylpentanol, 3-methylpentanol, 2-ethyl-butanol, isooctanol, n-decanol, or n-dodecanol, alone or copolymerized with ethylenically unsaturated monomers such as acrylic acid, methacrylic acid, acrylamide, methacrylamides, N-alkoxymethyl acrylamides, N-alkoxymethyl methacrylamides, N-t-butyl-acrylamide, itaconic acid, vinyl acetate, N-branched alkyl maleamic acids wherein the alkyl group has 10-24 carbon atoms, glycol diacrylates, or mixtures of these monomers; polyurethane elastomers; vinyl polymers such as polyvinyl alcohol, polyvinyl ethers, polyvinyl pyrrolidone, and polyvinyl acetate; urea formaldehyde resins; phenol formaldehyde resins, resorcinol formaldehyde resins; cellulose derivatives such as ethylcellulose, methylcellulose, nitrocellulose, cellulose acetate butyrate and carboxymethylcellulose; and natural gums such as guar, acacia, pectina, starch, destria, gelatin, casein, etc.

Other suitable pressure-sensitive adhesives include polyisobutylene pressure sensitive adhesives, rubber pressure-sensitive adhesives and silicone pressure-sensitive adhesives. The adhesives may also be compounded with tackifiers and stabilizers as is well-known in the art. Adhesives that are preferred for their active agent permeability include acrylic copolymer adhesives such as Avery Chemical Company's AS-351 HSX, preferably at a coating weight of between 25 and 35 g/m² . This pressure-sensitive adhesive is a cross-linkable polymer which provides a permanently tacky film having a total solids content of about 52%, Brookfield viscosity (LVT/Spindle No. 4/12RPM @ 25° C.) of from about 15,000 to 25,000 cps. at a weight per gallon of about 7.4 lbs. It can also be diluted with hexane or toluene to a desired solids and/or viscosity range, particularly for use in conventional coating equipment. Other such adhesives that can also be used for these purposes include an acrylic pressure-sensitive adhesive sold by National Adhesives under the designation DUROTAK 80-1054. This adhesive has a solids content of 47.5%, a viscosity of 3,000 cps., and plasticity (Williams) of 2.9 mm. It is generally used with a solvent system including ethyl acetate, heptane, isopropyl alcohol and toluene. Another such adhesive is sold by Monsanto under the designation GELVA Multipolymer Emulsion 2484, and comprises a stable aqueous acrylic emulsion pressure-sensitive adhesive having a solids content of 59% and a viscosity of 1,500 to 2,300 cps. Examples of other acrylic adhesives include Gelva 788 and 733 from Monsanto, PS-41 from C. L.-Hathaway, Vr-0833 from H. B. Fuller, Adcot 73A207A from Morton Chemical, Nos. 80-2404, 80-1054, 72-9056 and 72-9399 from National Starch, Nos. E-2015, E-2067 and E-1960 from Rohm & Haas, M-6112 from Uniroyal, Inc. and Daratak 74 L from W. R. Grace. Suitable rubber adhesives include Durotak 36-6172 from National Starch and Morstik 118 from Morton Chemical. An example of a suitable silicone adhesive is X7-4502 from Dow Corning.

The active ingredient permeable adhesive layers preferably contain some of the active ingredient when the device is placed on the skin. This provides an initial active ingredient presence at the skin or mucosa and eliminates delay in absorption of the active ingredient or in topical application, if that is desired. Thus, the active ingredient is immediately available to the host. The initial active ingredient presence may be due to the migration through the adhesive layer or layers and, if present, rate-controlling layer, or to an amount of the active ingredient mixed in with the active ingredient permeable adhesive layer or layers or rate-controlling layer during manufacture. Thus, while either or both the androgen and/or a permeation enhancer may be present in several of the laminate layers utilized, this may be the result of incorporation of the ingredients in only one of the layers, followed by migration of the ingredients to other layers. It should also be noted that the materials which can be used for creation of the active ingredient permeable adhesive layers may also be used as adhesive carrier layers for the androgen and any other drug or excipient to be administered with the androgen. When used as the drug reservoir, the patch may also include one or more rate controlling layers as discussed herein.

The width (i.e., surface area) and thickness of the permeable adhesive layer for contact with the skin or mucosa is that width and thickness which provides sufficient permeability to the active agent or active agent enhancer and a suitable surface area to allow the dosage rate desired to the skin or mucosa. These widths and thicknesses are conventional in the art and therefore need not be discussed in detail here.

The androgen carrier layer(s) may be monolithic polymeric active ingredient (androgen) carrier layers. Thus, in essence, these monolithic active ingredient carrier layers basically comprise a thermoplastic polymeric matrix which is admixed with the androgen and any other active agent, enhancer or excipient. The monolithic polymer matrix carrier layers may be made by blending the androgen with a matrix polymer in a common solvent and then evaporate the solvent to form a plastic film. The carrier layers of the present invention may also be formed by blending a thermoplastic matrix polymer with the active agent at an elevated temperature above which the polymer softens and melts, but below which the androgen in negatively affected, at which temperature the polymer is molten and fluid. This has been referred to as melt-blending. See, U.S. Pat. No. 5,662,926 the text of which is hereby incorporated by reference.

The androgen can also be included in both the carrier layer and rate-controlling layer. Such embodiments can include laminates that do not utilize an androgen enhancer, as well as laminates that have an androgen enhancer in one or more of the carrier layer, rate-controlling polymer layer, and androgen permeable adhesive layers. The present invention also includes embodiments in which androgen or the androgen enhancer are included in layers in which they have not been melt-blended, which layers may also be non-polymeric. Such layers are instead prepared and assembled into the laminate by conventional methods using prior art materials that are well-known to those of ordinary skill in the art. Laminates in accordance with the present invention, however, will at the least include a carrier layer of a thermoplastic matrix polymer melt-blended with an active agent, an active agent enhancer, or both.

In addition, the present invention further includes embodiments in which more than one carrier layer is present or more than one rate-controlling layer is present, or both, in any order, provided that at least one rate-controlling polymer layer, if present, is situated between a carrier layer and the skin or mucosa of the host. At least one carrier layer is melt-blended with an active agent, active agent enhancer, or both, otherwise the other layers may include an androgen, androgen enhancer, or both, or may be substantially free of an androgen or androgen enhancer. The androgen or androgen enhancer may be melt-blended with the other layers or combined with the other layers by conventional methods. The active agent and thermoplastic matrix polymer can be melt-blended in an extruder and then formed into the carrier layer by extrusion. Coextrution of various layers is also possible as is known in the art. When the enhancer is to be melt-blended with the carrier layer, rate-controlling polymer layer or active agent permeable adhesive layer, the enhancer should be an active agent enhancer heat stable at the melt temperature of the carrier polymer, rate-controlling polymer or active agent permeable adhesive into which it is to be melt-blended.

Suitable thermoplastic matrix polymers for the carrier layer include the class of elastomeric resins which are polyether block amides commercially designated by the trademark PEBAX. Another class of suitable thermoplastic matrix polymers is the thermoplastic polyurethanes. Of this class, the polyether polyurethanes are preferred. These include such commercial polyurethane compositions such as Dow Chemical Company's PELLETHANE, including its 2363-80 AE grade thereof; K. J. Quin's Q-THANE; B. F. Goodrich's ESTANE; Mobay Chemical Company's TXIN; and others.

Suitable thermoplastic matrix polymers also include various polyesters, such as the copolymers of various cyclic polyesters including DuPont's HYTREL, including its 4056 grade thereof, and General Electric's LOMOD both of which are copolymers of polyether prepolymers and polybutylene terephthalate and polyisobutylene terephthalate, respectively, as well as Eastman Chemical's PCCE. Other suitable polymers include ethylene methacrylic and acrylic acid copolymers such as ethylene methacrylic acid having the commercial designation NUCREL 699.

Suitable adhesive carriers also include any of the non-toxic polymers, particularly those of the type used to carry drugs for transdermal delivery including natural or synthetic elastomers, such as polyisobutylene, styrene, butadiene, styrene isoprene block copolymers, acrylics, urethanes, silicones, styrene butadiene copolymers, methyl acrylate copolymers, acrylic acid, polyacrylates, and polysaccharides such as, karaya gum, tragacanth gum, pectin, guar gum, cellulose, and cellulose derivatives such as methyl cellulose, propyl cellulose, cellulose acetate and the like, along with other substances known for use in transdermal preparations capable of forming a solid colloid that can adhere to skin and mucosa, used alone or in combination with other suitable carriers. A particularly preferred carrier is a bioadhesive for application to the dermis. The adhesive can be modified so as to adhere to the skin or mucosal tissue, depending on the intended application site. As stated above, preferred adhesives for application to the skin are bioadhesives. The term “adhesive” as used herein means a substance, inorganic or organic, natural or synthetic, that is capable of surface attachment to the intended application site. The term “bioadhesive” as used herein means an adhesive which attaches and preferably strongly attaches to a live or freshly killed biological surface such as skin or mucosal tissue upon hydration. Indeed, to qualify as a bioadhesive, a substance must be capable of maintaining adhesion in moist or wet in vivo or in vitro environments.

The strength of adherence can be measured by standard tests for measuring the force, e.g. in dynes per square centimeter, as disclosed in U.S. Pat. No. 4,615,697. Suitable bioadhesives include those prepared from optionally partially esterified polyacrylic acid polymers, including but not limited to, polyacrylic acid polymers lightly crosslinked with a polyalkenyl polyether such as those commercially available from B. F. Goodrich, Cincinnati, Ohio, under the trademarks Carbopol 934, 934P, 940 and 941.

Other suitable bioadhesives include natural or synthetic polysaccharides. The term “polysaccharide” as used herein means a carbohydrate decomposable by hydrolysis into two or more molecules of monosaccharides or their derivatives. Suitable polysaccharides include cellulose derivatives such as methylcellulose, cellulose acetate, carboxymethy-1cellulose, hydroxyethylcellulose and the like. Other suitable bioadhesives are pectin, a mixture of sulfated sucrose and aluminum hydroxide, hydrophilic poly-saccharide gums such as natural plant exudates, including karaya gum, ghatti gum, tragacanth gum, xanthan gum, jaraya gum and the like, as well as seed gums such as guar gum, locust bean gum, psillium seed gum and the like. In addition to the above ingredients, there may also be incorporated other additives selected from among the various pharmaceutically acceptable additives available to those skilled in the art. These additives include binders, stabilizers, preservatives and pigments.

The patch should be made of adhesives customary in medicine and other auxiliaries customary from pharmacopoeias (without skin-damaging or potentially skin-damaging properties). It should be possible for the patch to be charged with active ingredients to the highest possible level, without losing any of its adhesive strength, in order to generate uniformly high blood levels over the longest possible time.

When acrylate polymers are used, the acrylate polymer can be any desired homopolymer, copolymer or terpolymer comprising various acrylic acid derivatives. In such a preferred embodiment, the acrylic acid polymer makes up from about 2 to about 95% of the total weight in the total dermal composition, and preferably about 2 to about 90%. The amount of acrylate polymer depends on the amount and type of drug used which is incorporated in the medicament used. The acrylate polymers of this invention are polymers of one or more monomers of acrylic acids and other copolymerizable monomers. The acrylate polymers moreover comprise copolymers of alkyl acrylates and/or methacrylates and/or copolymerizable secondary monomers or monomers having functional groups. If the amount of each type added as a monomer is changed, the cohesive properties and solution properties of the resulting acrylate polymers can be changed. In general, the acrylate polymer comprises at least 50% by weight of an acrylate or alkyl acrylate monomer, 0 to 20% of a functional monomer which can be copolymerized with acrylate, and 0 to 40% of another monomer.

Acrylate monomers which can be used with acrylic acid and methacrylic acid are listed below: butyl methacrylate, hexyl acrylate, hexyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate and tridecyl methacrylate.

The following functional monomers which can be copolymerized with the above mentioned alkyl acrylates or methacrylates can be employed together with acrylic acid and methacrylic acid: maleic acid, maleic anhydride, hydroxyethyl acrylate, hydroxypropyl acrylate, acrylamide, dimethylacrylamide, acrylon-itrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate. See U.S. Pat. No. 5.683,711, the text of which is hereby incorporated by reference. Further details and examples of adhesive acrylates which are suitable for the invention are described in Satas' Handbook of Pressure Sensitive Adhesive Technology “Acrylic Adhesives”, 2nd edition, pp. 396-456 (D. Satas, Editor), Van Nostrand Reinhold, New York (1989).

Appropriate adhesive acrylates are commercially obtainable under the trade name Duro-Tak and include the polyacrylate adhesive. Appropriate polysiloxanes include pressure-sensitive silicone adhesives which are based on two main constituents: a polymer or adhesive and a tack-increasing resin. The polysiloxane adhesive is usually formulated with a crosslinking agent for the adhesive, typically a high molecular weight polydiorganosiloxane, and with the resin to provide a three-dimensional silicate structure via an appropriate organic solvent. Admixing of the resin to the polymer is the most important factor for modifying the physical properties of the polysiloxane adhesive. Sobieski et al., “Silicone Pressure Sensitive Adhesives”, Handbook of Pressure Sensitive Adhesive Technology, 2nd edition, pp. 508-517 (D. Satas, Editor), Van Nostrand Reinhold, New York (1989).

While the purpose of the topical, transdermal dosage forms of the present invention is the delivery of a selected group of androgens, other pharmaceutically active agents may be administered as well. These may include: psychoactive agents such as nicotine, caffeine, mesocarb, mefexamide, cannabinols such as THC, and the like, sedatives such as diazepam, mepiridine, uldazepam, tybamate, metaclazepam, tetrabarbitol and the like, antidepressants such as amitryptyline, imipramine desipramine, nialamide, melitracen, isocarboxazid, and the like, anticonvulsants such as phenobarbitol, carbamazepine, methsuximide, 2-ethyl-2-phenylmalonamide (PEMA), phenytoin and the like. Analgesics, including narcotic analgesics such as codeine, morphine, analorphine, Demerol and the like, and analgesics such as acetaminophen, aspirin, alprazolam and the like, antimicrobial agents such as sulconazole, siccanin, silver sulfadiazine, bentiacide, and the like, tranquilizers such as meprobamate and the like, antineoplastic agents such as sulfosfamide, rufocromomycin and the like, and antibiotic agents such as tetracycline, penicillin, streptozcin and the like.

The quantity of these other, non-androgen, active agents present in the transdermal patch, and indeed in the creams, lotions, gels, ointments, powders, salves and other transdermal formulations of the present invention is that quantity sufficient to provide a pharmaceutically or physiologically effective dosage rate of the active agent to a subject in need thereof. This quantity can be readily determined by those of ordinary skill in the art.

The relative proportion of androgen and any other drug in the dosage forms of the present invention will depend on a number of the factors discussed previously including the nature of the dosage form, the indication and the duration of administration, the flux of the androgen and the device etc. However, generally at least about 0.5% by weight of the dosage form will be androgen in accordance with the present invention. More preferably, the amount of androgen will range from between about 1.0 to about 80% by weight, most preferably from between about 5.0 to about 50% by weight.

The devices of the present invention optionally include a rate-controlling polymer layer which can be the active ingredient permeable adhesive layer. These adhesive or non-adhesive flow regulation layers can modify the rate at which the androgen is administered topically and, therefore, the flux. See, for example, U.S. Pat. Nos. 5,676,969 and 5,503,804, the text of which are hereby incorporated for reference. The polymers suitable for use as the rate-controlling polymer layer are conventional in the art and need not be discussed in detail here. Some preferred materials include, for example, polyethylene, polypropylene, ethylene vinyl acetate copolymer (EVA), copolyesters (e.g., HYTREL) and polyurethanes.

The rate of permeation of the active agent through the rate-controlling polymer layer depends on factors such as the affinity of the androgen for the polymer layer, molecular size of the androgen, polymeric structure of the carrier layer and the thickness of the layer. Therefore, the appropriate rate-controlling polymeric material and its thickness depend on the androgen used and the desired rate of permeation. The selection of a polymer layer and its thickness provides a means, if desired, for controlling the dosage rate to the skin or mucosa. An enhancer to promote the penetration of the androgen through the skin may also be included in either the carrier layer, rate-controlling polymer layer or the active agent permeable adhesive layers.

The enhancer may be incorporated into these layers by solvent blending or by melt-blending, i.e. by the same processes utilized to incorporate the androgen into either the carrier layer or the rate-controlling polymer layer. Suitable enhancers include those described in the above-cited U.S. Pat. No. 4,573,996, such as the following enhancers with a sufficiently high boiling point: monovalent, saturated and unsaturated aliphatic and cycloaliphatic alcohols having 6 to 12 carbon atoms such as cyclohexanol, lauryl alcohol and the like; aliphatic and cycloaliphatic hydrocarbons such as mineral oils; cycloaliphatic and aromatic aldehydes and ketones such as cyclohexanone; N, N-di (lower alkyl) acetamides such as N, N-diethyl acetamide, N, N-dimethyl acetamide, N-(2-hydroxyethyl) acetamide, and the like; aliphatic and cycloaliphatic esters such as isopropyl myristate and lauricidin; N, N-di (lower alkyl) sulfoxides such as decylmethyl sulfoxide; essential oils; nitrated aliphatic and cycloaliphatic hydrocarbons such as N-methyl-2-Pyrrolidone, Azone; salicylates, polyalkylene glycol silicates; aliphatic acids such as oleic acid and lauric acid, terpenes such as cineole, surfactants such as sodium lauryl sulfate, siloxanes such as hexamethyl siloxane; mixtures of the above materials; and the like.

In a preferred embodiment, the device contains a protective liner attached to the device at the surfaces to be adhered to the skin or mucosa, namely the active agent permeable adhesive layer and, if present, the peripheral adhesive layer. The protective liner may be made of the same materials suitable for use in the backing layer as discussed above. Such material is preferably made removable or releasable from the adhesive layers by, for example, by conventional treatment with silicon, Teflon or other suitable coating on the surface thereof. The removal of the device from the protective liner may also be provided by mechanical treatment of the protective liner, e.g., by embossing the protective liner.

The protective liner, however, can comprise various layers, including paper or paper-containing layers or laminates; various thermoplastics, such as extruded polyolefins, such as polyethylene; various polyester films; foil liners; other such layers, including fabric layers, coated or laminated to various polymers, as well as extruded polyethylene, polyethylene terephthalate, various polyamides, and the like.

A particularly preferred embodiment of the protective liner of the present invention includes a laminate of an outer foil layer and an inner layer of plastic, such as polyethylene or the like, which is rendered releasable not only by means of a siliconized coating, but which also includes an embossed or roughened surface. Embossment of this surface can be accomplished by a number of conventional methods. In general, preparation of embossed surfacing can be accomplished by the use of male-female tooling, preferably enhanced by the application of heat. The principle intention of this embossment process is to roughen the surface or render it uneven so that less than the entire surface will be in physical contact with the corresponding adhesive layer.

The actual pattern of embossment carried out can vary, and in some instances may involve embossment of large contiguous areas of the protective liner. Preferably, approximately 30% of the surface of the protective liner will thus be embossed. The particular design of the embossment, such as the production of a grainy texture or the like, is a matter of choice within the parameters discussed above. The presence of the embossed surface on the inner surface of the protective liner is thus extremely significant in preventing the protective liner from sticking or adhering to the adhesive layer or layers, which would cause the liner to fail to properly separate from the adhesive layer or layers when it is desired to use the device of the present invention. This ease of operation is an important element in commercialization of these devices.

The selection of a particular protective liner will also depend upon other ultimate requirements of the particular device in question, including whether there is a desire for a transparent or opaque liner, etc.

It can thus be seen that although substantially the entire surface of the protective liner is in contact with the adhesive layer or layers, the seal provided to the adhesive layer or layers by the protective liner is “peelable” or releasable, by merely pulling apart the edge of the protective liner. At the same time, when this is done, the adhesive layer or layers for contact with the skin or mucosa remain in contact with the surface of the carrier layer and the peripherally extended backing area, if present, because of the coefficient of adhesion between the adhesives and these layers vis-à-vis the coefficient of adhesion between these adhesive layers and the coated surface of the protective liner. See generally U.S. Pat. No. 5,662,926.

It is also possible to use a “bottom” layer which, if used, should be flexible enough to generally follow the contour of the area of the host where the device is to be applied. On the other hand, it should have enough strength and substance so as to serve its function of carrying the active agent carrying members without wrinkling, etc. The actual material from which the bottom layer can be produced can therefore include a variety of different materials.

Some suitable materials for this layer include, for example, polyethylene, polypropylene, polyvinylidine chloride, polyethylene terephthalate, polyesters, polyamides, and others, as well as laminates of two or more of these layers with each other or one or more of these layers with additional layers such as foil, paper, various fabrics, etc., but in these cases, preferably with the polymer layer on the inside, i.e., in contact with and thereby carrying the active agent carrying members. Therefore, in a preferred aspect of these embodiments of the invention, the bottom layer is a laminate of an outer foil layer and an inner layer of plastic, such as polyethylene or the like.

The backing layers of the active agent carrying members are disposed onto the bottom layer by one of the above-mentioned acrylic, natural rubber or synthetic rubber pressure-sensitive adhesive. The adhesive layer thickness is controlled in the conventional manner to insure that the active agent carrying members preferentially adhere to the skin or mucosa of the host over the bottom layer.

The various layers of the device of the present invention may be combined to form a laminate by methods conventional in the art. However, the present invention includes an inventive process for combining the active agent and a thermoplastic matrix polymer by melt-blending the two components, as well as an inventive process for combining polymer layers together by extrusion, preferably coextrusion.

The active agent and thermoplastic matrix polymer can be melt-blended using any art-recognized method for blending polymers with additives. Essentially, the thermoplastic matrix polymer is melt-blended with the active agent at a temperature above the softening point of the polymer using any conventional melt-blending apparatus including extruders, calendars, kneaders, sigma bladed mixers such as Brabender-type mixers, Banbury-type mixers and the like, preferably at a temperature between about 170° C. and about 200° C.

The active agent can also be melt-blended with the rate-controlling polymer by the above-described method. In addition, the active agent enhancer can also be melt-blended with either the thermoplastic matrix polymer or the rate-controlling polymer by the above-described method.

The carrier layers for the devices of the present invention can be formed directly from the resulting blend or die-cut from films formed therefrom. As such, the blends of thermoplastic matrix polymer and active agent of the present invention can be directly extruded, calendared, compression-molded, injection-molded, thermoformed or otherwise cast, by conventional solvent-free methods well-known to those of ordinary skill in the art. Alternatively, the blend of active agent and thermoplastic matrix polymer can first be formed by extrusion into pellets for storage, which pellets can subsequently be formed into the carrier layer by any of the above-mentioned forming methods.

The carrier layers of the present invention are preferably formed in compounded-extruders in which the active agent and thermoplastic matrix polymer can be melt-blended and the resulting melt-blend extruded into the above-mentioned pellets, or into a film from which carrier layers may be formed, or into the actual carrier layers. The entire process is carried out without dissolving the polymer, the active agent, or the active agent and polymer blend in a solvent for the polymer or active agent other than the optional compatible heat-resistant liquid carrier.

The monolithic carrier layer, once formed, can be immediately die-cut and combined on one surface with the backing layer. Alternatively, the layers can be combined prior to die-cutting. The backing layer is either laminated to the carrier layer by an adhesive layer, or by extruding the backing layer and carrier layer together. As will be readily understood by those of ordinary skill in the art, when the backing layer and carrier layer are extruded together without an active agent impermeable adhesive layer, then it is critical that the backing layer be formed from an active agent impermeable material.

The adhesive layer providing a means for affixing the device to the skin or mucosa for the host is applied to either the carrier layer and the extruded peripheral area of the backing layer, if present. If a rate-controlling polymer layer is affixed to the carrier layer, then any adhesive layer to be affixed to the carrier layer is applied to the rate-controlling polymer layer instead. Such adhesive layers can be applied either before or after the carrier layer and backing layer are laminated together.

Die-cutting, whenever mentioned herein, is carrier out by processes well-known in the laminating art.

As noted above, certain embodiments include a rate-controlling polymer layer affixed to the carrier layer on the surface to be applied to the skin or mucosa of a host. This polymer layer is either adhered to the carrier layer by an active agent permeable adhesive layer, or, this layer can also be extruded with the carrier layer, alone, or with the backing layer. As is well understood to those of ordinary skill in the polymer forming art, layers of the same or different polymers are conventionally extruded together. Two or more of the carrier layer, backing layer and rate-controlling polymer layer can be coextruded together in a single step. When all three layers are coextruded, the only adhesive layer required will adhere the rate-controlling polymer layer, and thus the laminate, to the skin or mucosa of the host.

The device, once formed, may be kept sealed in an air-tight pouch prior to use. The device of the present invention is used in the same manner as those devices which are conventional in the prior art. In most instances, the releasable protective liner attached to the skin-side surface of the adhesive layer or layers of the device for contact with the skin or mucosa of the host is removed and such surface of the adhesive layer or layers is applied to the desired area of the skin or mucosa.

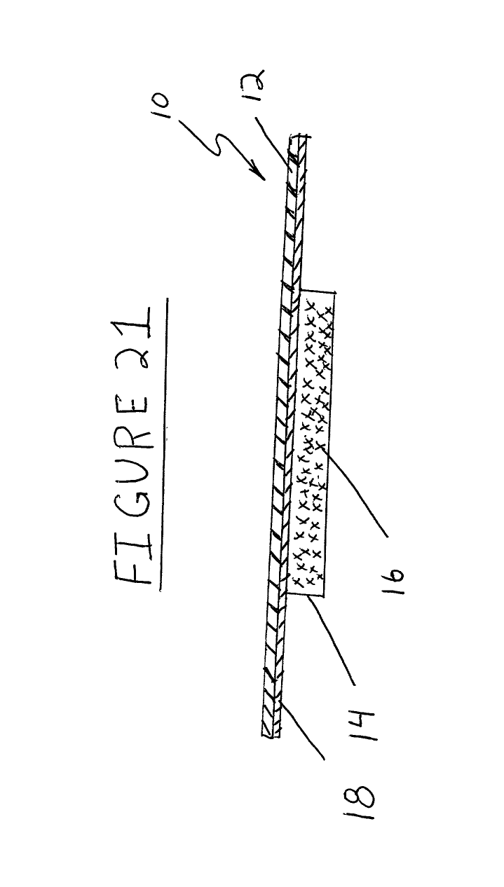

A transdermal patch can be very simple in construction. As illustrated in FIG. 21, a transdermal patch 10 can include an occlusive, non-androgen permeable backing layer 12 and a monolithic, androgen permeable carrier 14 containing the androgen 16. The carrier layer 14 can be affixed to the backing layer 12 using a suitable adhesive layer 18. That adhesive layer 18 may also be used to releasably affix the patch 10 to the skin of a subject and in particular, a human male. As described herein, other layers, not shown, can include rate controlling layers, peelable release layers and the like.

The flux of a specific formulation can be determined using a modified Franz diffusion cell as discussed herein. First, skin from female Sprague-Dawley rats (200-250 g body weight) can be obtained. The rats are anesthetized, the abdominal skin shaved and then excised. Excess fat and connective tissues are removed. The skin (1.77 cm²) is sandwiched between the two chambers of a modified Franz diffusion cell, with the surface of the skin facing the upper (donor) chamber of the cell. A measured amount of the androgen containing formulation is applied to the surface of the skin, so that the skin is completely covered. The lower (recipient) chamber is filled with sterile saline, which completely bathes the lower surface of the rat skin. The contents of the recipient chamber are constantly stirred with a magnetic stirrer. Aliquots of the saline in the recipient chamber is removed at predetermined intervals and analyzed by HPLC. The saline remains in constant contact with the skin at all times. The Franz cell is maintained at 37° C. for the duration of each test.

### EXAMPLES

### EXAMPLE 1

An in vitro modified Franz cell system as described in Example 2 was used to study the permeability of testosterone and MENT from various transdermal dosage forms through rat skin. Various topical cream, gel and patch formulations were tested. The concentration of testosterone and MENT in all of the gels and creams described herein was 2 mg per gram of gel or cream. The following formulations were tested:

(A) Commercial Gel Base Formulations Tested:

(1) KY Jelly Lot: 28G787A

Ortho-McNeil Pharmaceutical, Inc., Raritan, N.J. 08869

Preparation Tested: 10% Ethyl Alcohol added

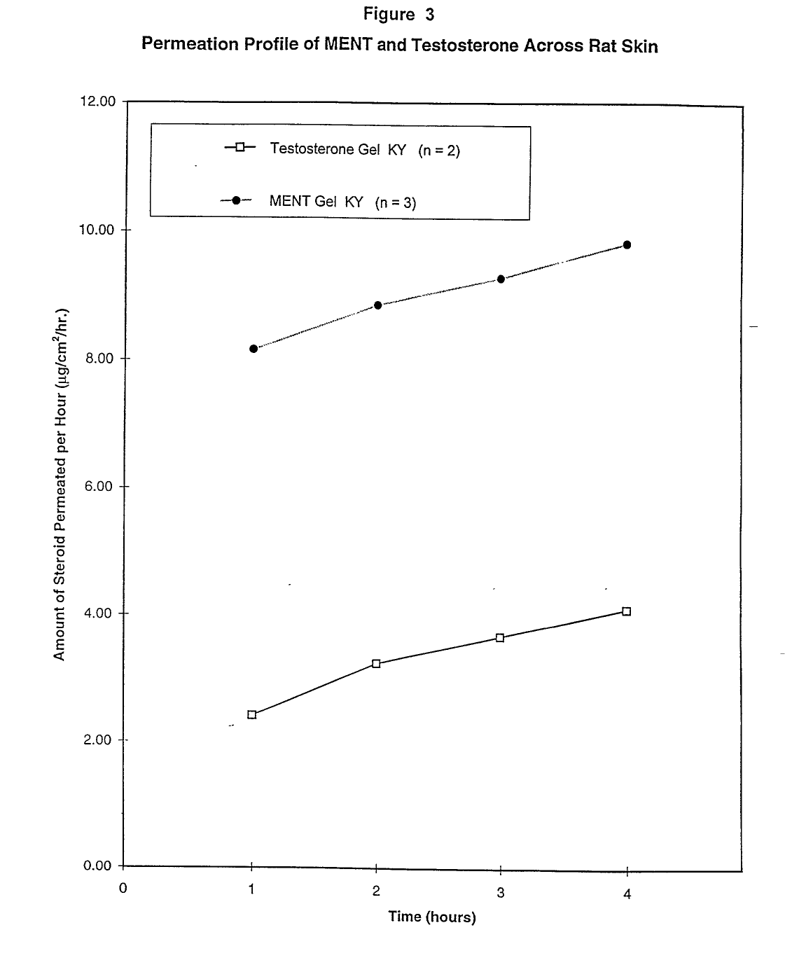

(Results illustrated in FIG. 3)

(2) Pharmacist Value Lubricating Jelly

Distributor: Taro Pharmaceuticals, Hawthorne, N.Y. 10532

Preparation Tested: 10% Ethyl Alcohol added

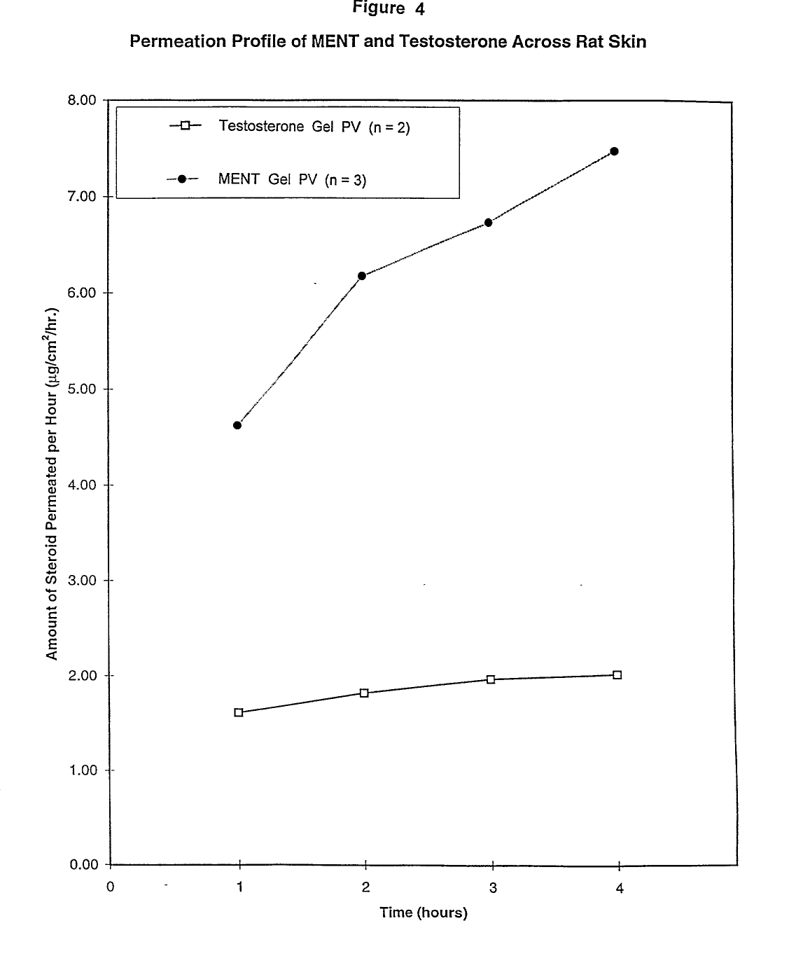

(Results illustrated in FIG. 4)

In each of these gel bases, 2 mg of MENT or testosterone were mixed per gram of base along with 10% ethyl alcohol by weight of the finished gel. These formulations were prepared by measuring an amount of the base material and blending therein the appropriate amount of drugs and alcohol until homogeneity was reached.

(B) Transdermal Patch

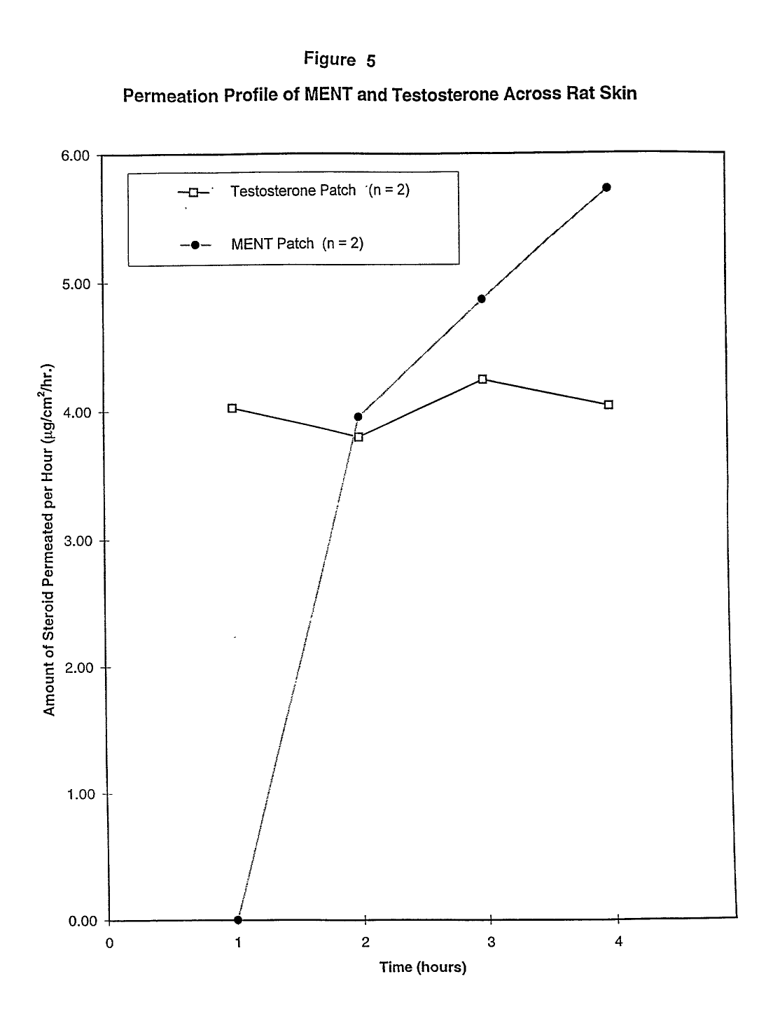

Transdermal patches, containing either testosterone or MENT were made from a silicone elastomer (NuSil R-2602, Nusil Silicone Technology, Carpinteria, Calif. 93013). The drug load was 25% (w/w). The drug was measured and blended with an appropriate amount of the elastomer. A catalyst, stannous octoate, was then added and the formulation was mixed. The material was then injected into three centimeter diameter molds and allowed to polymerize to form a monolithic carrier material. The disks were then adhered to an impervious backing of polyethylene having an outer metallic coating. Adhesion was provided by using a silicone medical grade adhesive. The resulting patches contained neither permeation enhancers nor adhesives on the drug releasing surface. Such patches could be adhered to the skin of a subject by use of a backing layer having an extended flange which surrounds the drug containing carrier. The flange could have a suitable adhesive disposed on the skin contacting surface, such that the resulting structure would resemble an adhesive bandage. (Results illustrated in FIG. 5)

(C) Cream Formulations Tested:

(1) Commercial Cream Base A

Lot: 471. Medco Lab., Inc., Sioux City, Iowa 51103

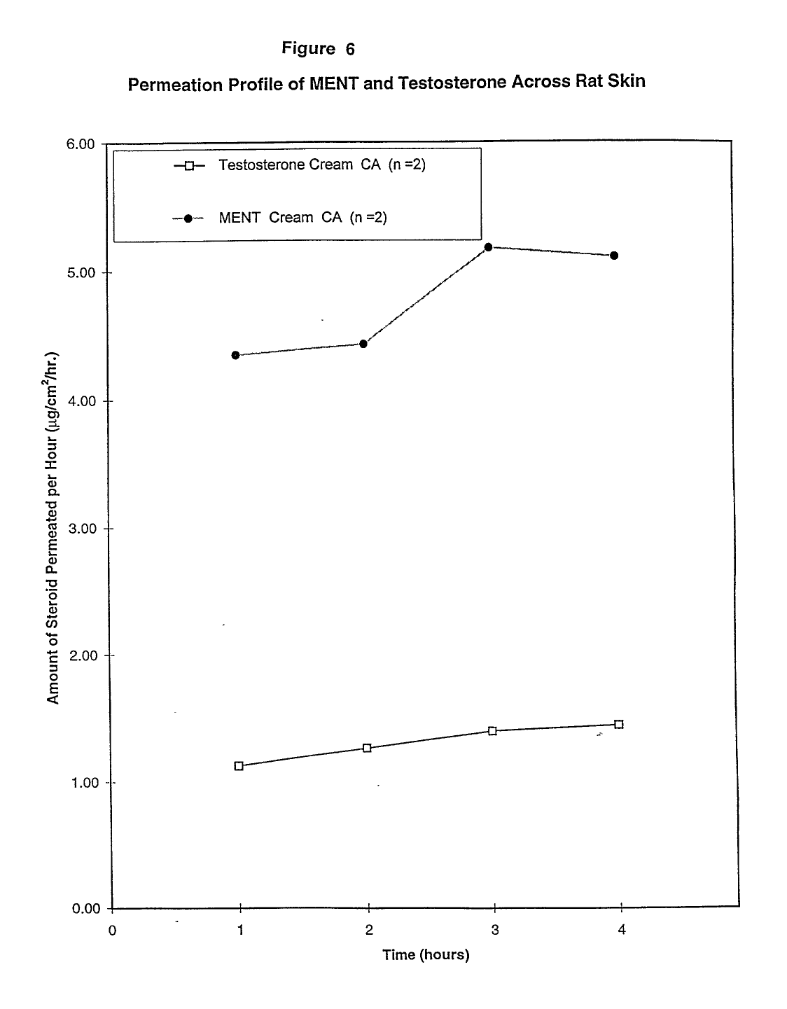

10% Ethyl Alcohol added (Results illustrated in FIG. 6)

(2) Commercial Cream Base B-Aquaphilic Ointment Lot: 1305. Medco Lab., Inc., Sioux City, Iowa 51103

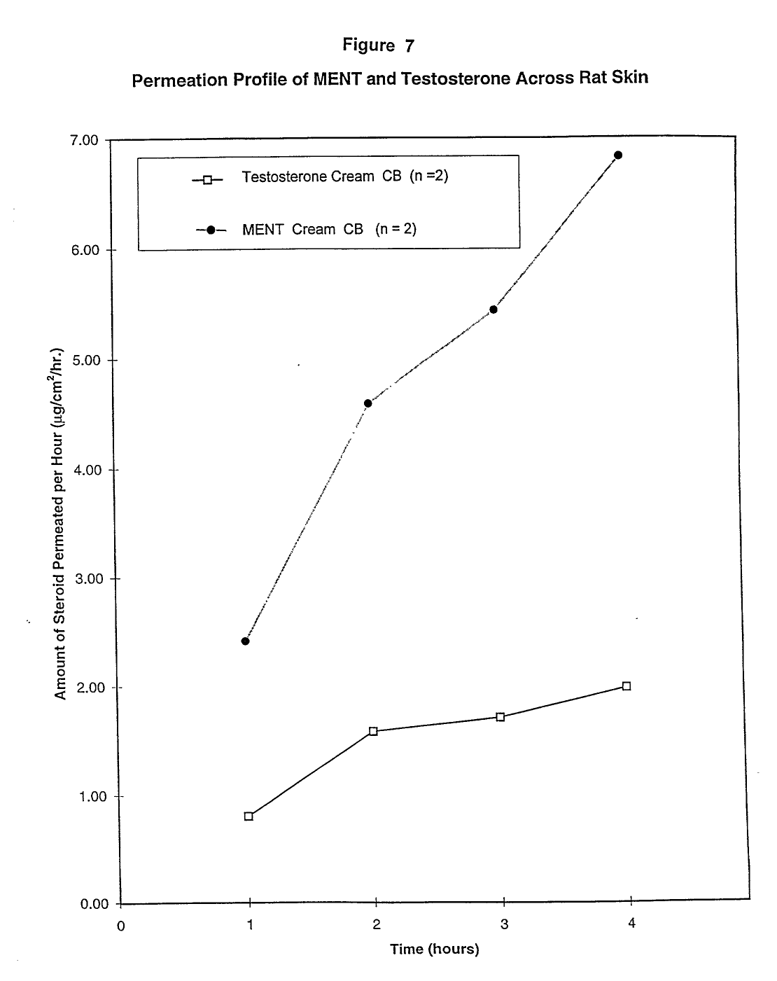

10% Ethyl Alcohol added (Results illustrated in FIG. 7)

(3) Formulation CBR:

| | | |
|---|---|---|
| Stearyl Alcohol | 24 | g |
| White Petrolatum | 20 | g |
| Sodium Lauryl Sulfate | 1 | g |
| Mineral Oil | 2 | g |
| Propylene glycol | 12 | g |
| Ethyl acetate | 4 | g |
| Isopropyl Alcohol | 5 | g |
| Ethyl Alcohol | 10 | g |
| Water qs | 100 | g |

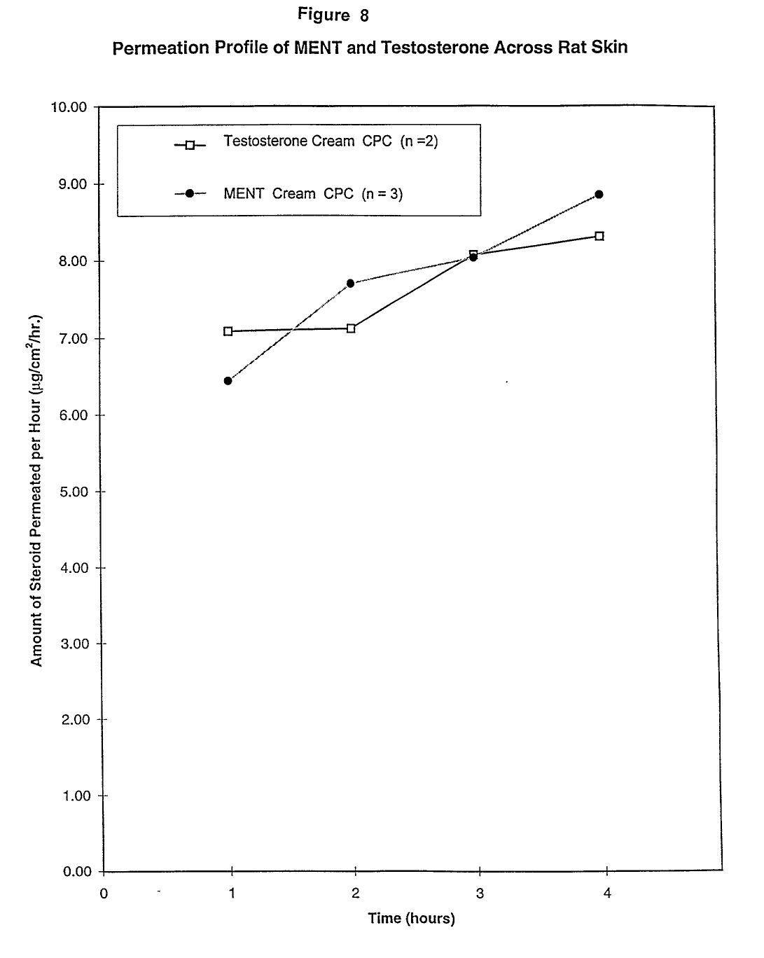

(Results illustrated in Figure 8)

The cream base for Formulation CBR was produced by mixing all of the ingredients in a single reaction vessel using medium agitation. For formulation of the therapeutic topical creams in accordance with the present invention, each of these three cream bases were measured and the appropriate amount of drug was blended therein to homogeneity.

(D) Gel Formulations

| | Formulation | | | | |
|---|---|---|---|---|---|
| | O | D | F | P | T |
| Component | (g) | (g) | (g) | (g) | (g) |
| Methyl cellulose | 1.0 | 1.0 | 0.2 | 0 | 0 |
| Carbopol | 0.3 | 0.3 | 0.8 | 1.0 | 0.8 |
| Benzyl alcohol | 0.9 | 0.9 | 0.9 | 0.9 | 0.9 |
| Propylene glycol | 35.0 | 25.0 | 25.0 | 23.0 | 23.0 |
| Isopropyl alcohol | 10.0 | 10.0 | 10.0 | 10.0 | 10.0 |
| Ethanol | 0 | 10.0 | 10.0 | 12.0 | 12.0 |
| Water q s | 100.0 | 100.0 | 100.0 | 100.0 | 100.0 |

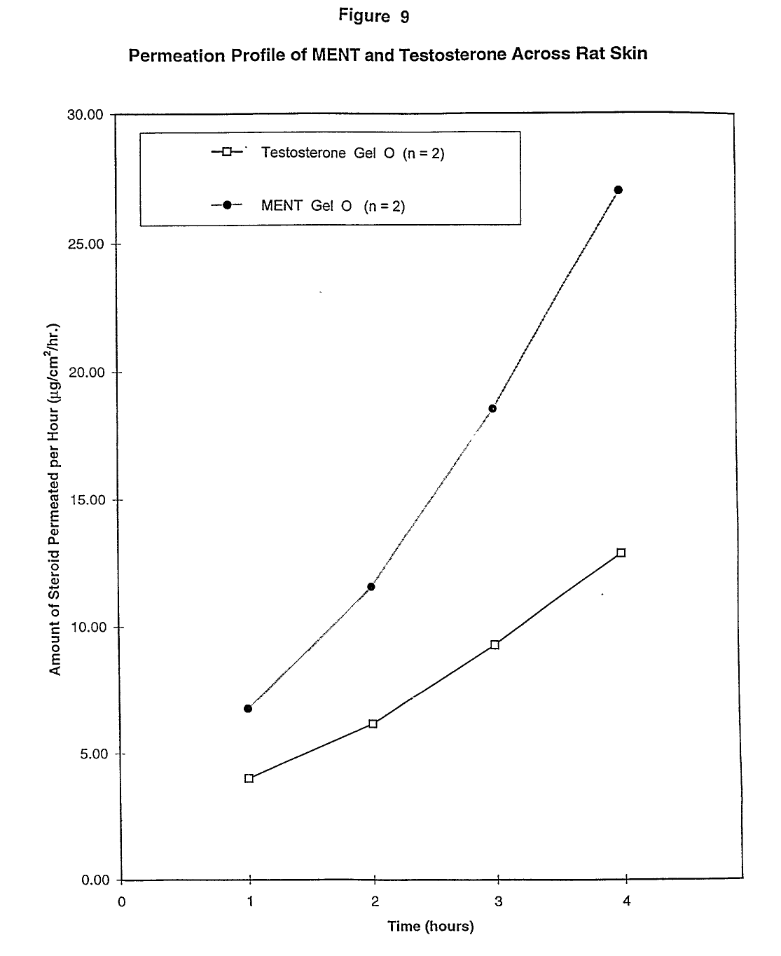

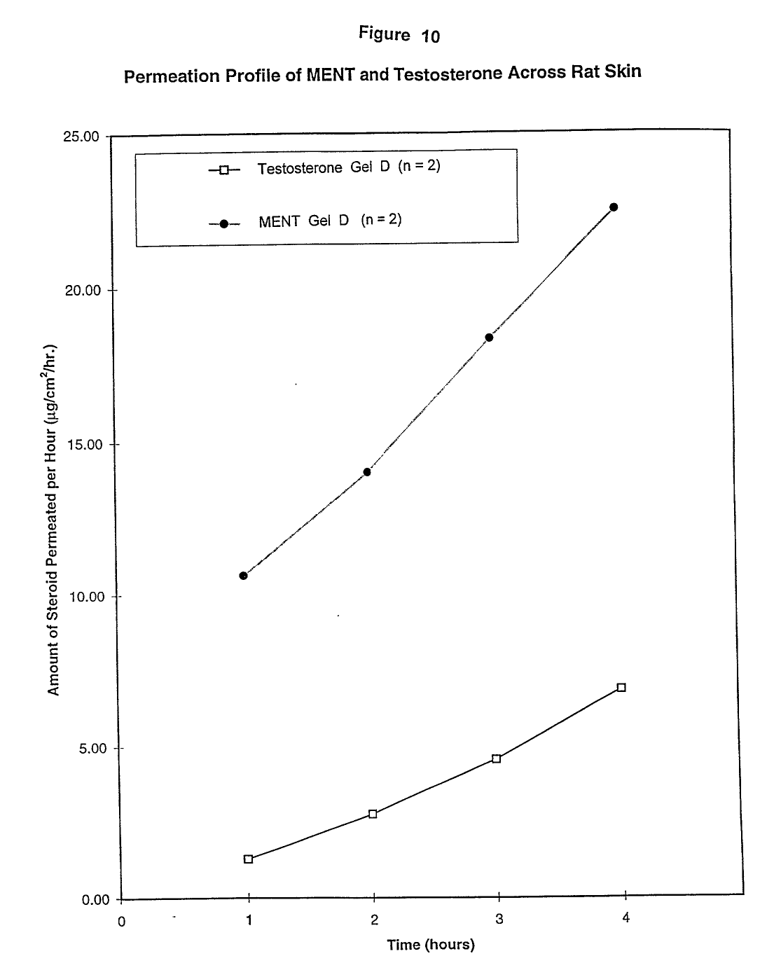

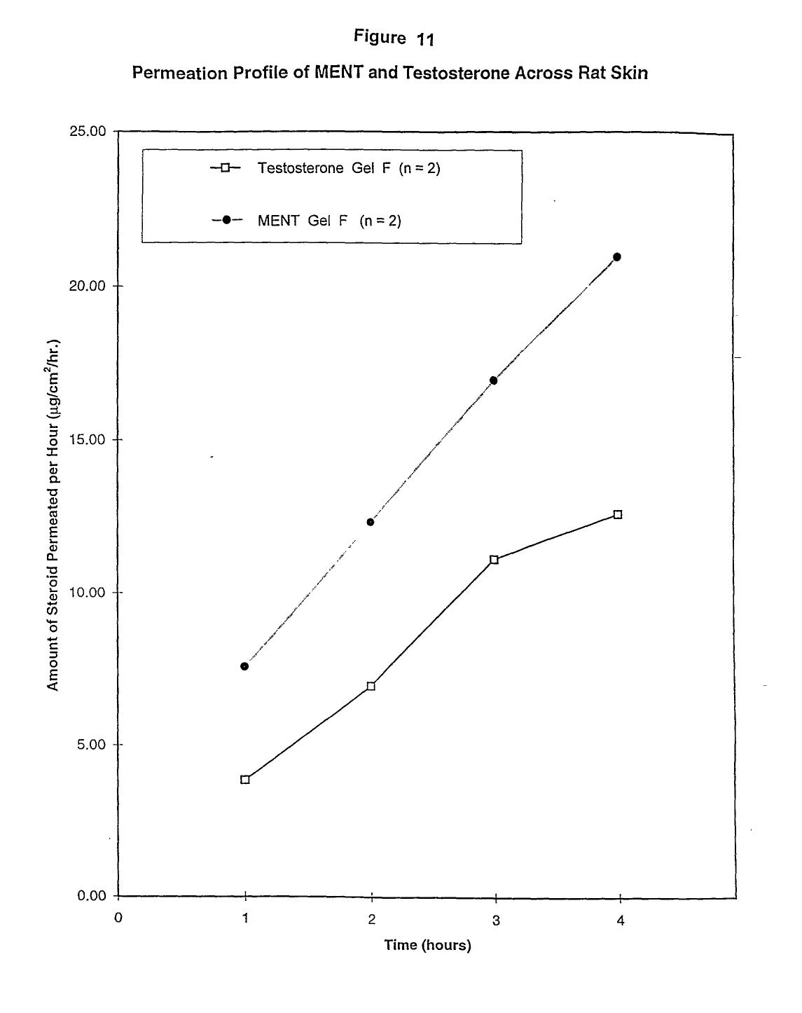

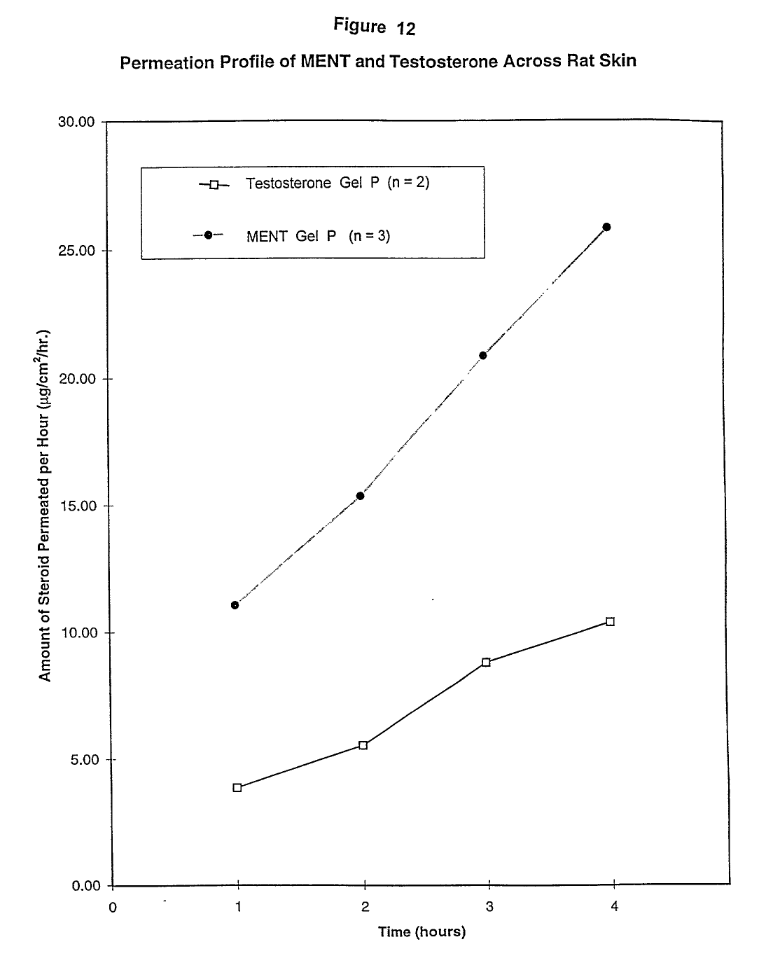

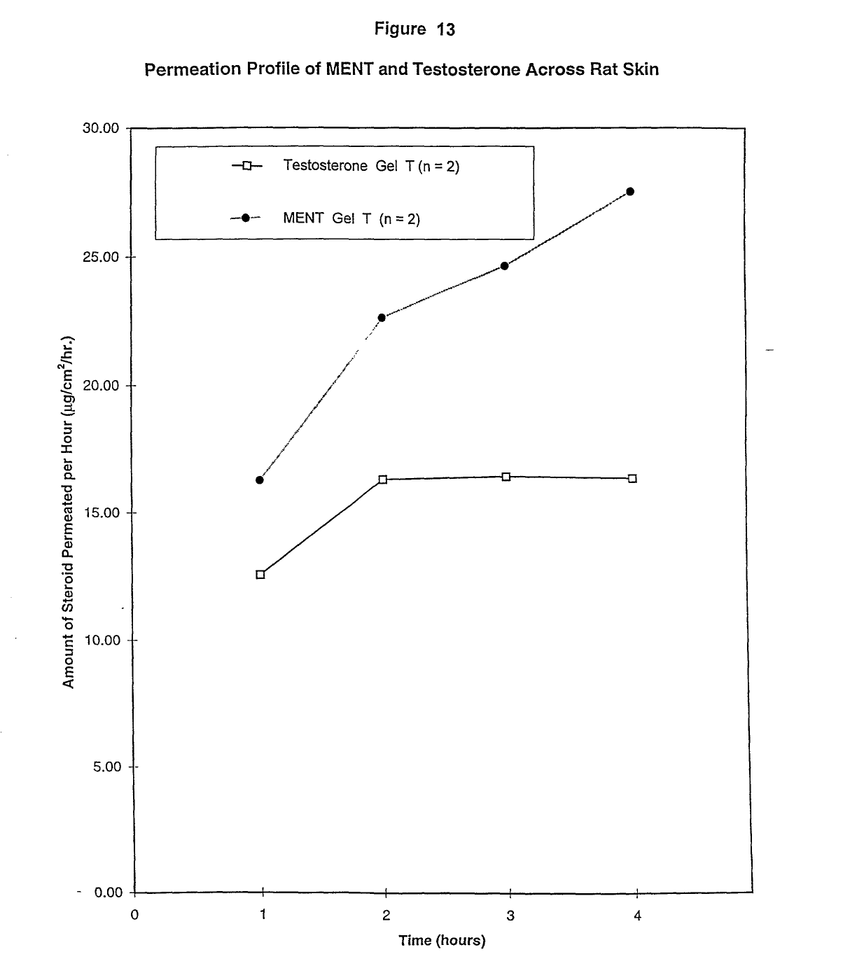

Note:
1N Sodium Hydroxide was used to adjust pH to 6.5-7.5.
(The results for gel formulation O are illustrated in Figure 9, for formulation D see Figure 10, for formulation F see Figure 11, for formulation P see Figure 12, for formulation T see Figure 13.) These gels were produced as described in accordance with Example 2.

The results illustrated in FIGS. 3 through 13 indicate that in one cream foundation, MENT and testosterone permeated through rat skin at similar rates (FIG. 8). Of course, a close examination of FIG. 8 reveals that at most points of comparison, the MENT formulation provided a superior flux to that of the identical formulation including testosterone. In particular, transdermal dosage forms in accordance with the present invention preferably have, overall, a greater flux than the identical formulation using testosterone and this cream, as well as the other formulations identified herein, demonstrate same. In all other formulations (1 patch, 2 creams and 7 gels) tested, MENT permeated through rat skin at considerably higher concentrations and flux rates than that of testosterone (FIGS. 3 to 7 and 9 to 13). Indeed, in certain dosage forms, the flux of MENT was more than double that of testosterone. (See FIGS. 3, 4, 6, 7, 10, 11 and 12.)

Section II: In vivo Studies

The bioavailability of three transdermal MENT gel formulations (2 mg MENT/g gel) (formulations O, F and T) were studied in rabbits. Three New Zealand white rabbits weighing 3.5-4.5 kg were used in each group. To each animal, 0.2 g gel (0.4 mg MENT) or 0.4 g gel (0.8 mg MENT) was applied to 5×5 cm or 10×10 cm area of shaved skin for three consecutive days. On days 1 and 3, blood samples were collected at 0, 1, 2, 4 and 8 hours after application of the gel. Serum MENT levels were determined by radioimmunoassay.

TABLE 1
Bioavailabiity of MENT in Rabbits

| | Dose | | Area under the curve |
|---|---|---|---|
| Formulation | (mg) | Day | (ng/hr/mL) |
| O | 0.4 | 1 | 3.4 |
| | | 3 | 3.8 |
| | 0.8 | 1 | 12.5 |
| | | 3 | 10.4 |
| F | 0.4 | 1 | 27.5 |
| | | 3 | 8.2 |
| | 0.8 | 1 | 41.3 |
| | | 3 | 31.0 |
| T | 0.4 | 1 | 28.6 |
| | | 3 | 28.3 |
| | 0.8 | 1 | 51.8 |
| | | 3 | 52.8 |

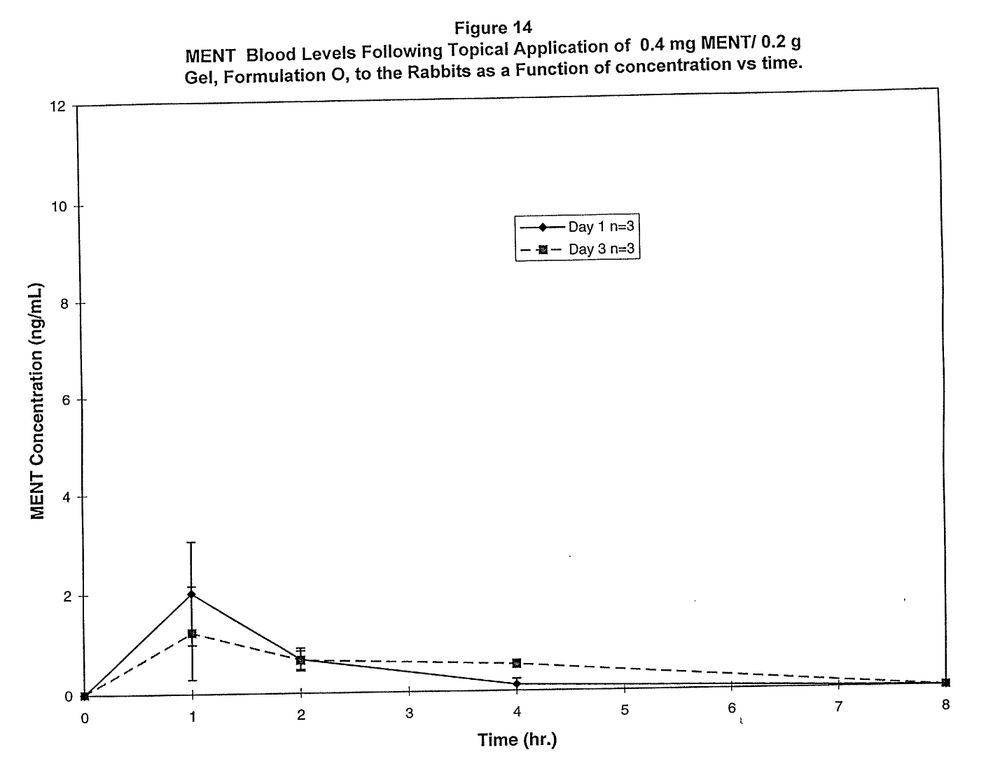

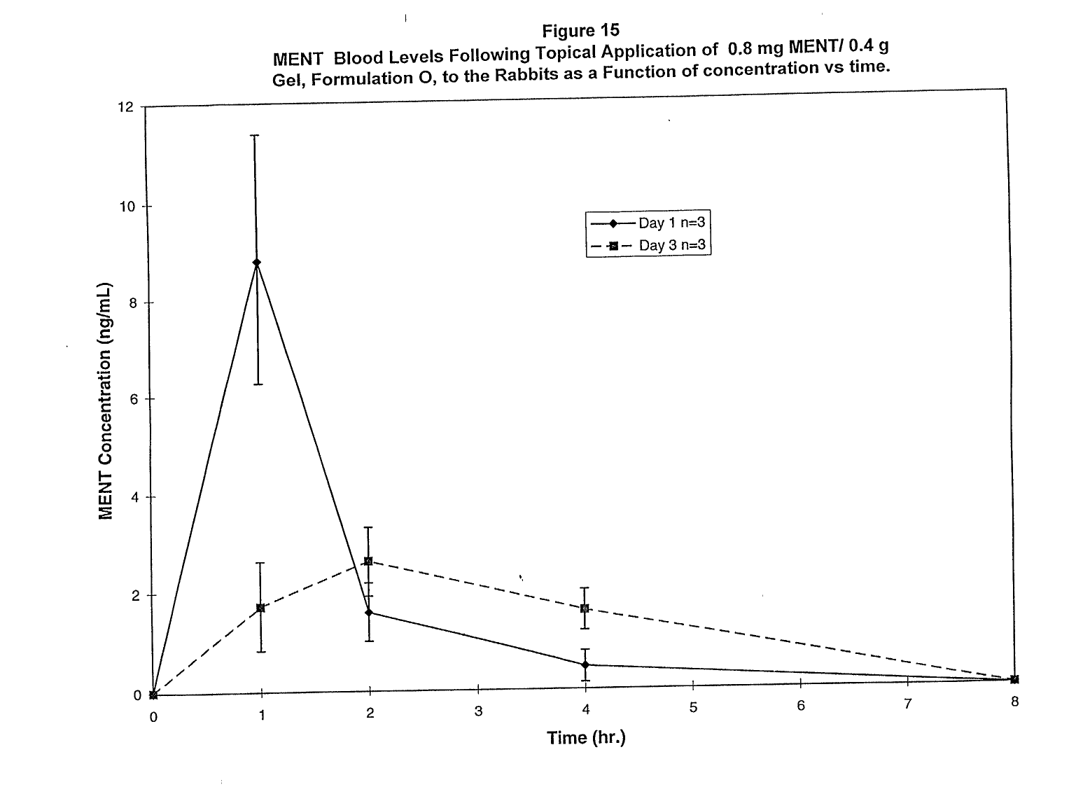

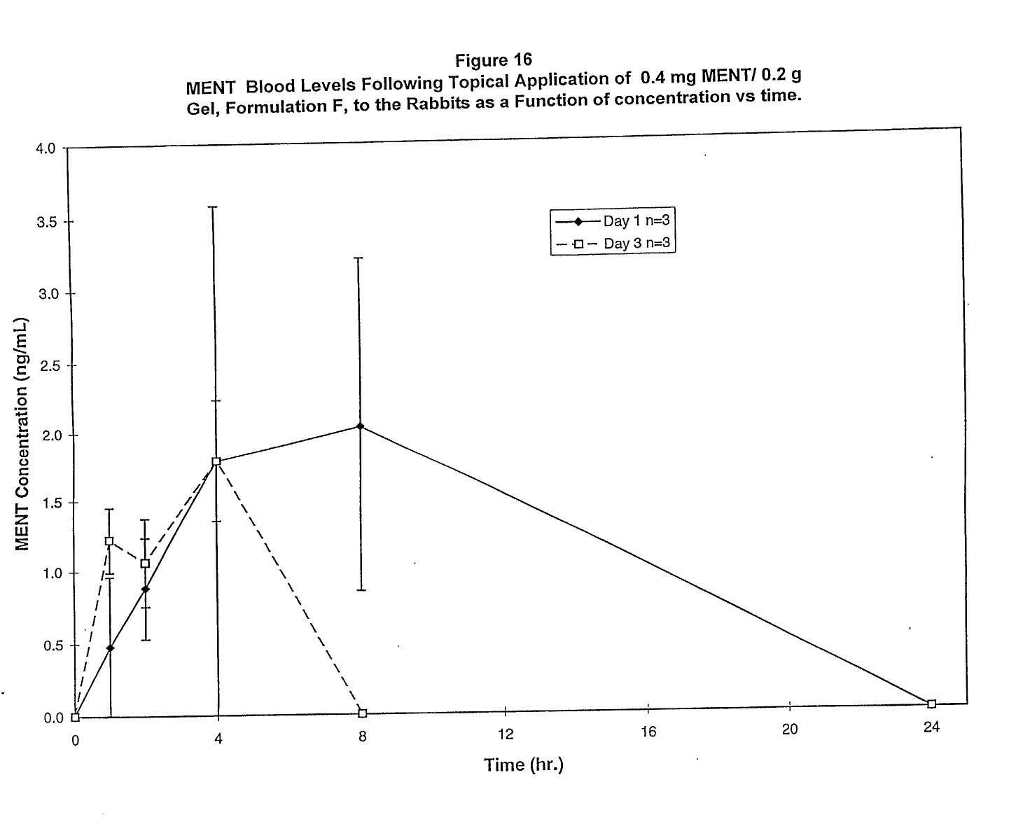

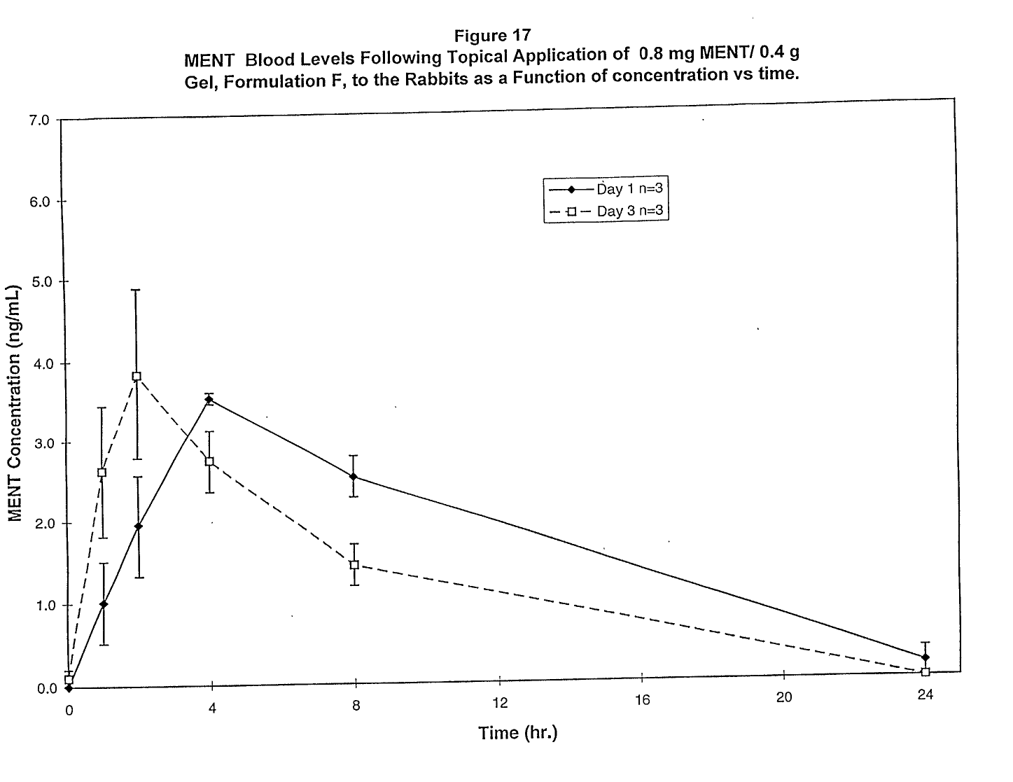

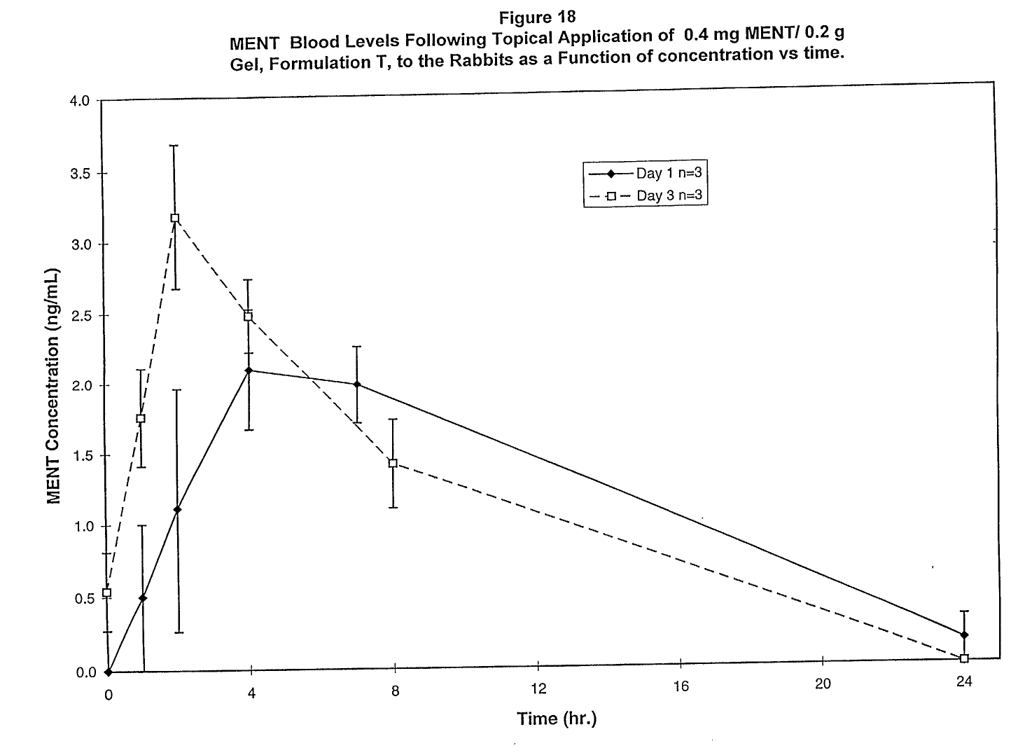

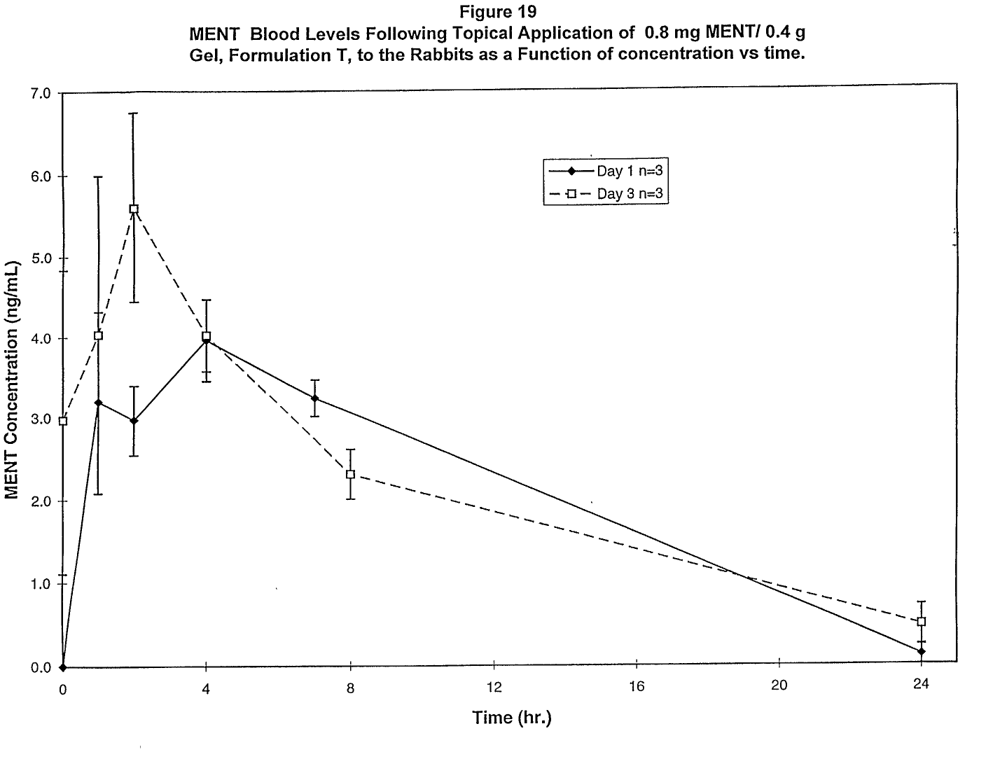

The results indicated that MENT in all formulations permeated through rabbit skin and gave measurable serum levels (FIGS. 14 to 19). The bioavailability of MENT was also dose dependent (Table 1).

A topical gel was prepared containing either MENT or testosterone (“T”).
For each gram of topical gel prepared, the composition was as follows:

| | | |
|---|---|---|
| 1. MENT (or T) | 2 | mg |
| 2. Propylene glycol | 230 | mg |
| 3. Ethyl alcohol | 120 | mg |
| 4. Isopropyl alcohol | 100 | mg |
| 5. Benzyl alcohol | 9 | mg |
| 6. Carbopol 934 | 8 | mg |
| 7. IN Sodium Hydroxide | 70 | mg |
| 8. Water | 461 | mg |
| Total | 1000 | mg |

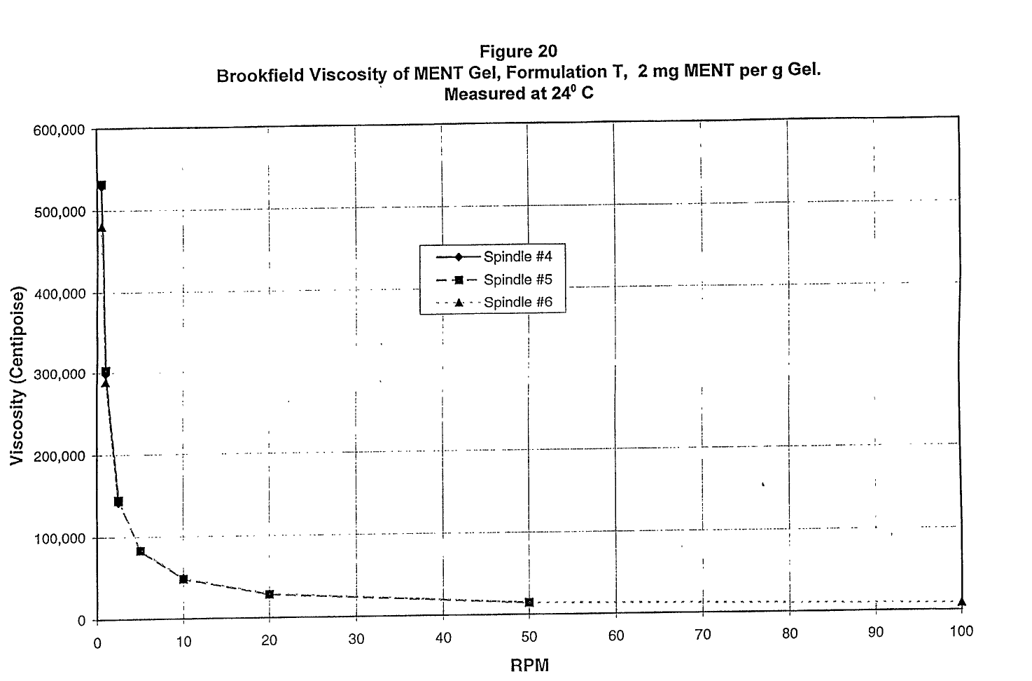

The gel (nearly identical to formulation T from Example 1) was produced by taking all of the aqueous components and mixing them under medium agitation in one vessel and all the organic, non-aqueous materials and mixing them in a separate vessel. The organic mixture and aqueous mixture were then mixed together using medium agitation provided by a paddle mixer. Of course, a lightning mixer or magnetic stirrer could also be used. The viscosity profile of the gel produced is illustrated in FIG. 20. Steroid permeation (in vitro) across Rat Skin for each of these gel formulations were tested using a two chambers of a modified Franz diffusion cell as previously described. Skin from female Sprague-Dawley rats (200-250 g body weight) were used in these studies. The rats were anesthetized, the abdominal skin was shaved and excised. Excess fat and connective tissues were removed. The skin (1.77 cm²) was sandwiched between the two chambers of a modified Franz diffusion cell, with the surface of the skin facing the upper (donor) chamber of the cell. Approximately 0.5 gm of gel (1 mg steroid) was applied to the surface of the skin, so that the skin was completely covered with the gel. The lower (recipient) chamber was filled with 11.4 ml of sterile saline, which completely bathed the lower surface of the rat skin. The contents of the recipient chamber was constantly stirred with a magnetic stirrer during the experiment. Aliquots of 200 μL of the saline in the recipient chamber were removed at intervals of 1 h for 4 h; and analyzed by HPLC for either MENT on T. The saline remained in constant contact with the skin at all times. The Franz cell was maintained at 37° C. for the duration of the test.

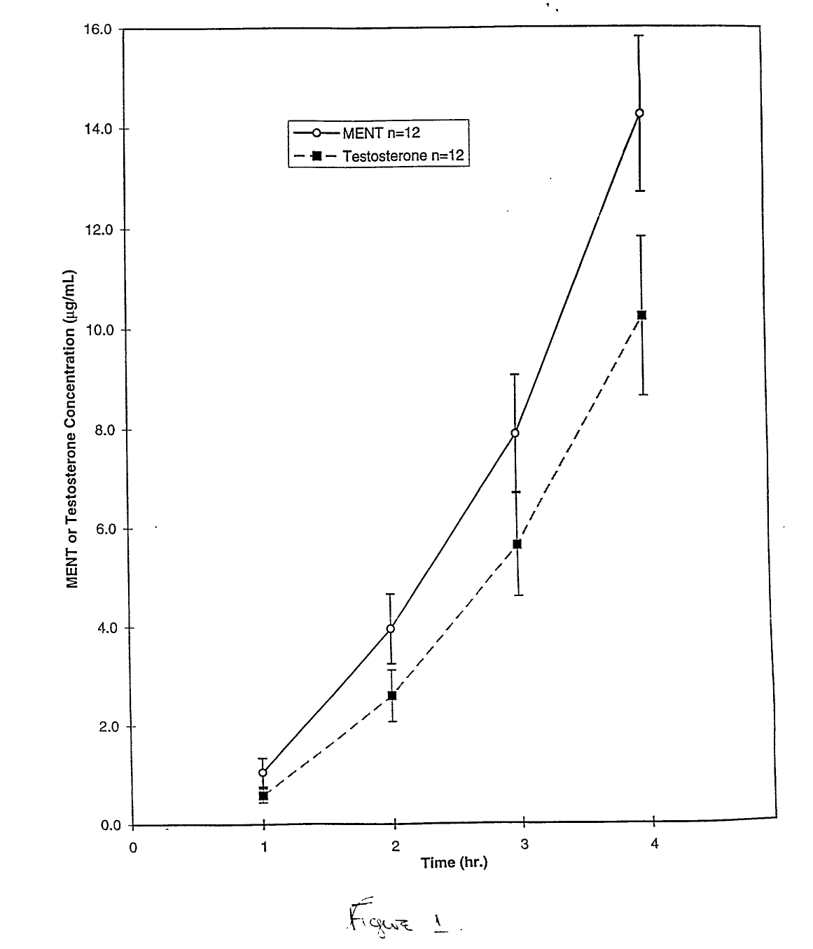

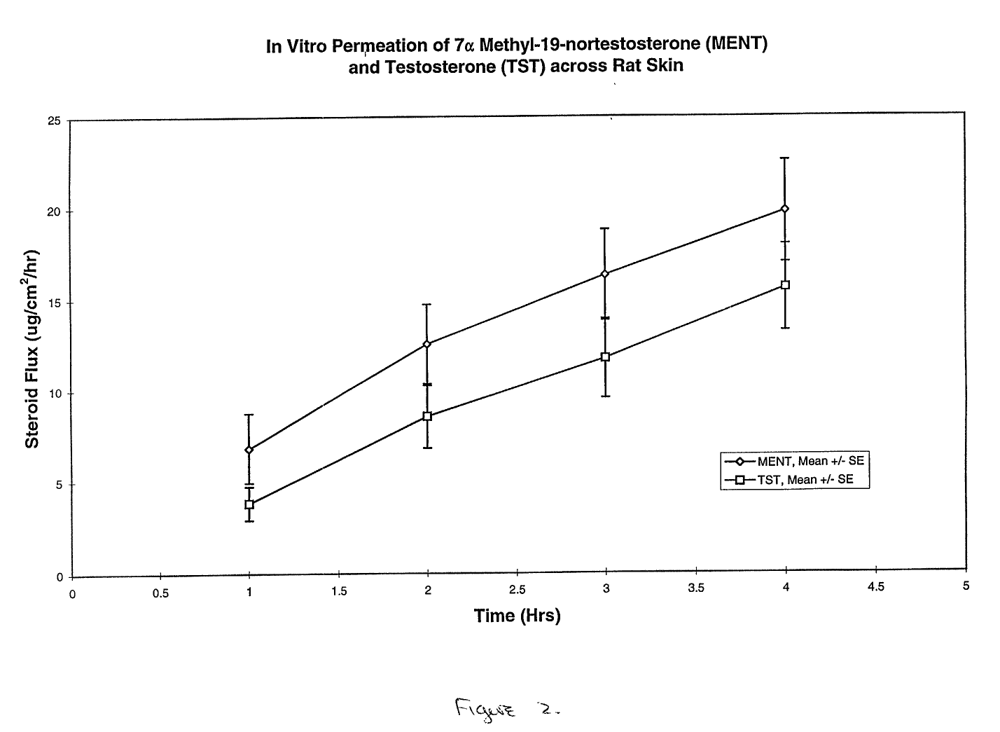

FIG. 1 illustrates the resulting penetration of MENT and T across rat skin from these gel formulations. FIG. 2 shows the steroid flux of both MENT and T gel formulations. Both Figures clearly demonstrate that MENT penetrates the skin at a faster rate than T, and that the flux of MENT was greater at each time period studied.

We claim:

1. A transdermal dosage form comprising: a non-5α-reducible androgen in therapeutically effective amount, dispersed within a pharmaceutically-acceptable, transdermal carrier.

2. The transdermal dosage form of claim 1 wherein said non-5α-reducible androgen is a 7α- modified androgen.

3. The transdermal dosage form of claim 2 wherein said 7α-modified androgen is a 7α-methyl-19-nortestosterone.

4. The transdermal dosage form of claim 1 wherein said androgen is provided in an amount of between about 0.5 to about 90% by weight of the dosage form.

5. The transdermal dosage form of claim 4 wherein said androgen is provided in an amount of between about 1.0 to about 80% by weight of the dosage form.

6. The transdermal dosage form of claim 5 wherein said androgen is provided in an amount of between about 5.0 to about 50% by weight of the dosage form.

7. The transdermal dosage form of claim 1 wherein said dosage form has a flux greater than that exhibited by an equal amount of testosterone when administered through an otherwise identical transdermal dosage form.

8. The transdermal dosage form of claim 1 wherein said pharmaceutically-acceptable transdermal carrier is an ointment.

9. The transdermal dosage form of claim 1 wherein said pharmaceutically-acceptable transdermal carrier is a gel.

10. The transdermal dosage form of claim 1 wherein said pharmaceutically-acceptable transdermal carrier is a cream.

11. The transdermal dosage form of claim 1 wherein said pharmaceutically-acceptable transdermal carrier is a lotion.

12. The transdermal dosage form of claim 1 wherein said pharmaceutically-acceptable carrier is a powder.

13. The transdermal dosage form of claim 1 wherein said pharmaceutically-acceptable carrier is a spray.

14. The transdermal dosage form of claim 1 wherein said pharmaceutically-acceptable carrier is a transdermal patch.

15. A transdermal dosage form comprising: between about 0.5 and about 10 mg of 7α-methyl-19-nortestosterone per day of use dispersed in a pharmaceutically-acceptable transdermal carrier said 7α-methyl-19-nortestosterone being present in an amount of about 0.5 to about 90% by weight relative to the weight of the pharmaceutically-acceptable transdermal carrier.
